SEQUENCE LISTING (4 sequences)

SEQ ID NO: 1 - protein, 15 aa, artificial sequence
  description of artificial sequence synthetic peptide
REVQSKIGYGRQIIS

SEQ ID NO: 2 - protein, 24 aa, artificial sequence
  description of artificial sequence synthetic peptide
ELMTGELPYSHINNRDQIIFMVGR

SEQ ID NO: 3 - protein, 21 aa, artificial sequence
  description of artificial sequence synthetic peptide
TGSLPYSHIGSRDQIIFMVGR

SEQ ID NO: 4 - protein, 18 aa, artificial sequence
  description of artificial sequence synthetic peptide
GIVLYELMTGELPYSHIN

(19) United States
(12) Patent Application Publication
(10) Pub. No.: US 2010/0036094 A1
(43) Pub. Date: Feb. 11, 2010

(54) Polypeptide Markers for the Diagnosis of Alzheimer's Disease

(75) Inventor: Harald Mischak, Sehnde (DE)

Correspondence Address: OHLANDT, GREELEY, RUGGIERO & PERLE, LLP, ONE LANDMARK SQUARE, 10TH FLOOR, STAMFORD, CT, 06901, US

(73) Assignee: MOSAIQUES DIAGNOSTICS AND THERAPEUTICS AG, Hannover (DE)

(21) Appl. No.: 11/887,603

(22) PCT Filed: Apr. 5, 2006
(86) PCT No.: PCT/EP2006/061336

§ 371 Date: Sep. 28, 2007

(30) Foreign Application Priority Data
Apr. 6, 2005 (EP) 05102705.0

(51) Int. Cl. C07K 14/00; H01J 49/26

(52) U.S. Cl. 530/350; 204/451; 250/282

(57) ABSTRACT

A method for the diagnosis of Alzheimer's disease, comprising the step of determining the presence or absence of at least one polypeptide marker in a sample, wherein the polypeptide marker is selected from markers 1 to 50 (frequency markers), or determining the amplitude of at least one polypeptide marker selected from markers 51 to 279 (amplitude markers)

### BACKGROUND

1. Field of the Disclosure

The present disclosure relates to the use of the presence or absence of one or more peptide markers in a sample from a subject for the diagnosis of Alzheimer's disease and to a method for the diagnosis of Alzheimer's disease, wherein the presence or absence of the peptide marker or markers is indicative of the existence of Alzheimer's disease.

2. Discussion of the Background Art

Neuropsychiatric Diseases of advanced age More than 70% of the aged population suffer from dementia diseases, whose most frequent cause is Alzheimer's disease, the proportion of the diseased increasing approximately exponentially from less than 20% among the 65-69 year old to more than 30% among the over 85 year old. All in all, the number of afflicted persons in Germany is currently about one million; by the year 2050, they would have increased to 2.5 million when the same prevalence is assumed. Almost 2% of the previously healthy older people are afflicted by dementia per year; in Germany, this corresponds to more than 200,000 incidences per year. Already today, one third of all people reaching the age of 65 must expect to develop a dementia in the further course of ageing. Minor symptoms of a possibly approaching dementia or the earliest stages of the disease that cannot yet be reliably diagnosed, in the form of the still not well defined disorder picture of minimal cognitive impairment (MCI), occur far more frequently than manifest dementia. From field studies, prevalence rates of from 10 to 20%, in individual cases also more than 30%, are often reported. However, the prediction of dementia is still associated with a high uncertainty in the individual case.

### Alzheimer's Disease

Alzheimer's disease is also referred to as Alzheimer's or “dementia of Alzheimer's type”. The term “dementia” means a decline in mental faculties. Alzheimer's disease is characterized mainly by an initial weakness of memory, which increases in the course thereof and may lead to a total loss of power of judgment and personality. Immediately after strokes, Alzheimer's disease is the most frequent severe disorder of brain function in age.

With the increase of life expectancy, the disease that today bears Alzheimer's name was diagnosed more and more frequently in the developed countries. Severe impairment of memory, paranoia, insomnia and restlessness are the most important signs; however, these symptoms may also occur in other diseases singly or in combination, such as in the case of age-related dementia and stroke.

About one million dementia patients are living in Germany, two third thereof suffering from Alzheimer's. The risk of becoming afflicted with Alzheimer's disease increases with increasing age. According to estimations, about five percent of the population of over 65 years and about 20 percent of the population of over 80 years are concerned in the Western countries. Because females live longer than males on average, their risk of becoming afflicted with Alzheimer's disease is thus clearly higher. Although Alzheimer's disease is considered an age-related disease, rare hereditary forms of the condition may onset as early as from the thirtieth year.

Although Alzheimer's disease is still incurable presently, the possibilities of treatment have improved in recent years. The earlier Alzheimer's disease is recognized and treated, the better is the chance of slowing down the disease process.

The disease mostly begins several decades before the first symptoms occur. Depositions of protein fragments, so-called amyloids, are formed in the brain. The amyloids are distinguished into microscopically small fibers, i.e., fibrils, and spherical depositions, i.e., plaques. Evidently, these depositions prevent nerve cells from communicating with each other. In the course of time, the nerve cells then die off in those regions of the brain that are involved in the development of memory, speech and reasoning powers.

Only in exceptional cases, the disease is triggered by gene alterations and breaks out in younger ages already. This may happen, for example, when the genetic information of the amyloid precursor protein (APP) is damaged. Harmful cleavage products of APP, which form the most important component of plaques, are increasingly formed. Alterations in other genes, the presenilins, have similar effects. They increase the activity of enzymes that degrade APP, and therefore also accelerate the formation of lumps in the brain.

The most important genetic risk factor is a molecule (ApoE) which is involved in the transport of cholesterol in the blood. The genetic information for ApoE occurs in three variants. One variant (ApoE4) increases the risk of disease by four to five times on statistical average, another variant (ApoE2) lowers the risk.

### Diagnosis

Although simple tests are reported from time to time, Alzheimer's disease can be established with absolute certainty only after a patient's death when the typical depositions can be found in the brain. In practice, it is important to make as certain as possible a diagnosis as early as possible.

Typical symptoms of Alzheimer's disease are:
impairment of short-term memory
difficulty in reasoning
speech disorders
depressions
restricted power of judgment
delusions
changes of personality

A deterioration of the short-term memory as a first symptom mostly can be observed at the age of 60 to 70 years already. The power of concentration and cognitive power decline, speech disorders occur, tiredness increases. The symptoms of depression frequently occur in the initial phase. They are accompanied by changes of behavior, such as confusion, anxiety, restlessness and aggressiveness. They can no longer cope with everyday skills, such as dressing, preparing meals or shopping, and finally they lose control of their physical functions. In the final stage, the patients often become silent, bedridden and completely depend on other persons' help.

As already described, there is no functioning early detection and no reliable diagnosis for Alzheimer's. Thus, there has been a need to find a process and method for a diagnosis of Alzheimer's disease that is as little as possible invasive, quick and inexpensive.

In Electrophoresis 26 (2005), 1476-1487, Wittke et al. describe the use of markers in human urine in CE-MS coupling and their suitability in principle for the diagnosis of Alzheimer's disease. From today's point of view, it is seen that the markers found therein have only a small significance.

The object of the present disclosure is to overcome at least some of the mentioned drawbacks of the prior art, especially to provide markers that have an improved significance over those of the prior art.

### SUMMARY OF THE DISCLOSURE

Surprisingly, it has now been found that particular peptide markers in combination in a sample from a subject can be used for the diagnosis of Alzheimer's disease. Consequently, the present disclosure relates to the use of the presence or absence of at least three polypeptide markers in a sample from a subject for the diagnosis of Alzheimer's disease, wherein said polypeptide marker is selected from polypeptide marker Nos. 1 to 279 as characterized by the molecular masses and migration times as stated in Table 1.

TABLE 1
Polypeptide markers for the diagnosis of Alzheimer's disease and their
molecular masses and migration times:

| | Mass | Migration |
|---|---|---|
| No. | [g/mol] | time [min] |
| 1 | 1073.35 | 34.96 |
| 2 | 1073.35 | 35.9 |
| 3 | 1110.31 | 37.67 |
| 4 | 1111.3 | 34.97 |
| 5 | 1196.36 | 36.37 |
| 6 | 1265.63 | 27.22 |
| 7 | 1322.44 | 36.2 |
| 8 | 1387.07 | 20.42 |
| 9 | 1390.49 | 36.26 |
| 10 | 1423.57 | 29.85 |
| 11 | 1453.55 | 30.47 |
| 12 | 1490.43 | 35.48 |
| 13 | 1494.62 | 30.23 |
| 14 | 1819.86 | 23.51 |
| 15 | 1927.02 | 21 |
| 16 | 2128.97 | 26.03 |
| 17 | 2214.22 | 26.48 |
| 18 | 2313.26 | 30.32 |
| 19 | 2423.19 | 27.03 |
| 20 | 2428.11 | 27.16 |
| 21 | 2521.33 | 27.86 |
| 22 | 2644.31 | 21.1 |
| 23 | 2706.37 | 28.3 |
| 24 | 3068.46 | 30.03 |
| 25 | 3079.61 | 37.02 |
| 26 | 3153.38 | 35.65 |
| 27 | 3229.79 | 25.02 |
| 28 | 3438.77 | 23.49 |
| 29 | 3481.8 | 30.62 |
| 30 | 3567.61 | 23.88 |
| 31 | 3685.85 | 25.47 |
| 32 | 3921.84 | 29.49 |
| 33 | 4036.3 | 24.92 |
| 34 | 4038.22 | 20.53 |
| 35 | 4093.96 | 21.04 |
| 36 | 4145.22 | 24.51 |
| 37 | 4372.3 | 19.37 |
| 38 | 4377.95 | 30.81 |
| 39 | 4496.98 | 19.35 |
| 40 | 4516 | 19.37 |
| 41 | 4540.4 | 22 |
| 42 | 4586.98 | 19.38 |
| 43 | 4596.23 | 19.6 |
| 44 | 4810.95 | 19.77 |
| 45 | 5227.91 | 25.08 |
| 46 | 5582.16 | 24.3 |
| 47 | 6402.73 | 22.3 |
| 48 | 7749.32 | 19.97 |
| 49 | 7760.02 | 28.72 |
| 50 | 10671.25 | 28.92 |
| 51 | 824.48 | 19.92 |
| 52 | 840.49 | 19.92 |
| 53 | 846.38 | 25.96 |
| 54 | 897.41 | 25.86 |
| 55 | 911.3 | 34.1 |
| 56 | 950.57 | 24.13 |
| 57 | 973.26 | 35.47 |
| 58 | 984.45 | 26.32 |
| 59 | 988.57 | 35.65 |
| 60 | 1013.43 | 25.18 |
| 61 | 1016.3 | 35.53 |
| 62 | 1041.58 | 36.79 |
| 63 | 1053.52 | 25.25 |
| 64 | 1055.61 | 25.7 |
| 65 | 1082.55 | 23.75 |
| 66 | 1082.65 | 26.15 |
| 67 | 1083.5 | 27.14 |
| 68 | 1083.55 | 25.61 |
| 69 | 1085.63 | 36.39 |
| 70 | 1094.61 | 25.99 |
| 71 | 1130.37 | 35.19 |
| 72 | 1130.63 | 27.07 |
| 73 | 1135.56 | 27.03 |
| 74 | 1150.61 | 26.55 |
| 75 | 1158.62 | 27.02 |
| 76 | 1183.66 | 27.49 |
| 77 | 1186.56 | 27.21 |
| 78 | 1217.56 | 21.8 |
| 79 | 1229.62 | 27.38 |
| 80 | 1250.61 | 27.59 |
| 81 | 1276.43 | 35.7 |
| 82 | 1283.4 | 35.91 |
| 83 | 1300.67 | 29.7 |
| 84 | 1306.74 | 22.16 |
| 85 | 1333.44 | 35.93 |
| 86 | 1342.44 | 35.43 |
| 87 | 1347.71 | 28.94 |
| 88 | 1349.67 | 28.25 |
| 89 | 1360.67 | 28.06 |
| 90 | 1386.68 | 28.62 |
| 91 | 1388.74 | 27.97 |
| 92 | 1399.44 | 36.06 |
| 93 | 1404.73 | 29.49 |
| 94 | 1409.63 | 22.11 |
| 95 | 1449.7 | 22.04 |
| 96 | 1451.72 | 28.84 |
| 97 | 1464.7 | 30.57 |
| 99 | 1475.77 | 30.05 |
| 100 | 1499.74 | 29.89 |
| 101 | 1519.76 | 31.87 |
| 102 | 1535.74 | 30 |
| 103 | 1542.78 | 23.24 |
| 104 | 1550.67 | 27.47 |
| 105 | 1552.61 | 30.78 |
| 106 | 1590.81 | 32.46 |
| 107 | 1596.83 | 30.32 |
| 108 | 1614.84 | 31.54 |
| 109 | 1623.65 | 31.1 |
| 110 | 1628.8 | 20.75 |
| 111 | 1636.79 | 22.71 |
| 112 | 1659.81 | 27.41 |
| 113 | 1692.91 | 31.38 |
| 114 | 1703.89 | 33.01 |
| 115 | 1727.92 | 32.13 |
| 116 | 1732.88 | 31.36 |
| 117 | 1738.68 | 32.16 |
| 118 | 1741.83 | 30.56 |
| 119 | 1756.89 | 19.81 |
| 120 | 1771.97 | 30.02 |
| 121 | 1800.02 | 25 |
| 122 | 1816.98 | 33.53 |
| 123 | 1820.06 | 32.23 |
| 124 | 1841.77 | 36.26 |
| 125 | 1849.68 | 18.31 |
| 126 | 1850.96 | 31.97 |
| 127 | 1867.72 | 33.07 |
| 128 | 1869.92 | 31.28 |
| 129 | 1873 | 21.26 |
| 130 | 1900.07 | 24.19 |
| 131 | 1914.07 | 25.67 |
| 132 | 1915.72 | 34.24 |
| 133 | 1917.03 | 24.99 |
| 134 | 1955.95 | 27.81 |
| 135 | 2021.11 | 21.85 |
| 136 | 2041.99 | 32.56 |
| 137 | 2053.18 | 20.54 |
| 138 | 2072.15 | 27.04 |
| 139 | 2077.07 | 25.44 |
| 140 | 2085.12 | 33.75 |
| 141 | 2140.12 | 20.56 |
| 142 | 2166.09 | 27.84 |
| 143 | 2168.66 | 34.88 |
| 144 | 2179.03 | 25.31 |
| 145 | 2278.11 | 26.17 |
| 146 | 2283.18 | 26.31 |
| 147 | 2314.3 | 22.71 |
| 148 | 2320.43 | 23.21 |
| 149 | 2327.24 | 34.9 |
| 150 | 2341.12 | 26.74 |
| 151 | 2343.24 | 34.95 |
| 152 | 2368.24 | 21.24 |
| 153 | 2377.25 | 28.07 |
| 154 | 2390.26 | 27.33 |
| 155 | 2392.73 | 35.48 |
| 156 | 2409.28 | 27.64 |
| 157 | 2410.3 | 30.62 |
| 158 | 2448.39 | 21.07 |
| 159 | 2475.21 | 27.09 |
| 160 | 2475.27 | 24.74 |
| 161 | 2486.04 | 35.76 |
| 162 | 2500.33 | 19.99 |
| 163 | 2521.36 | 19.26 |
| 164 | 2522.35 | 27.9 |
| 165 | 2527.42 | 19.79 |
| 166 | 2549.29 | 27.66 |
| 167 | 2584.37 | 35.6 |
| 168 | 2684.32 | 21.38 |
| 169 | 2698.41 | 22.52 |
| 170 | 2799.17 | 25.07 |
| 171 | 2816.31 | 28.74 |
| 172 | 2851.42 | 27.7 |
| 173 | 2923.51 | 36.73 |
| 174 | 2982.72 | 19.97 |
| 175 | 3019.54 | 24.72 |
| 176 | 3173.68 | 22.95 |
| 177 | 3192.67 | 37.66 |
| 178 | 3215.61 | 28.53 |
| 179 | 3218.4 | 30.31 |
| 180 | 3221.59 | 22.7 |
| 181 | 3279.66 | 25.32 |
| 182 | 3282.34 | 36.05 |
| 183 | 3298.42 | 36.17 |
| 184 | 3302.68 | 23.31 |
| 185 | 3324.7 | 21.62 |
| 186 | 3386.77 | 22.21 |
| 187 | 3401.73 | 23.48 |
| 188 | 3408.76 | 26.04 |
| 189 | 3462.68 | 21.25 |
| 190 | 3466.75 | 23.9 |
| 191 | 3524.8 | 31.72 |
| 192 | 3529.84 | 23.7 |
| 193 | 3595.77 | 23.89 |
| 194 | 3605.4 | 21.53 |
| 195 | 3614.82 | 22.01 |
| 196 | 3644.79 | 29.39 |
| 197 | 3645.25 | 26.69 |
| 198 | 3650.74 | 19.9 |
| 199 | 3672.71 | 21.9 |
| 200 | 3677.74 | 24.3 |
| 201 | 3685.86 | 22.21 |
| 202 | 3687.78 | 20.58 |
| 203 | 3693.93 | 27.15 |
| 204 | 3741.63 | 20.1 |
| 205 | 3768.77 | 31.84 |
| 206 | 3782.94 | 22.68 |
| 207 | 3788.04 | 25.24 |
| 208 | 3792.76 | 27.25 |
| 209 | 3819.82 | 32.74 |
| 210 | 3881.87 | 24.56 |
| 211 | 3903.08 | 25.71 |
| 212 | 3905.8 | 27.72 |
| 213 | 3929.73 | 23.07 |
| 214 | 3934.76 | 24.02 |
| 215 | 3951.07 | 22.77 |
| 216 | 4005.54 | 22.57 |
| 217 | 4087.12 | 26.92 |
| 218 | 4112.78 | 24.46 |
| 219 | 4131.68 | 22.54 |
| 220 | 4164.86 | 25.11 |
| 221 | 4192.99 | 34.95 |
| 222 | 4203.13 | 26.14 |
| 223 | 4218.31 | 20.6 |
| 224 | 4232.32 | 32.35 |
| 225 | 4250 | 27.68 |
| 226 | 4278 | 25.3 |
| 227 | 4301.93 | 21.26 |
| 228 | 4307.09 | 31.58 |
| 229 | 4349.06 | 28.17 |
| 230 | 4384.07 | 19.8 |
| 231 | 4428.79 | 32.86 |
| 232 | 4464.64 | 21.54 |
| 233 | 4468.61 | 23.47 |
| 234 | 4510.97 | 28.56 |
| 235 | 4548.03 | 25.95 |
| 236 | 4562 | 29.31 |
| 237 | 4562 | 29.31 |
| 238 | 4564.18 | 26.01 |
| 239 | 4583.84 | 24.2 |
| 240 | 4637.81 | 27.82 |
| 241 | 4730.71 | 19.63 |
| 242 | 4749.41 | 21.81 |
| 243 | 4750.06 | 19.53 |
| 244 | 4890.59 | 23.27 |
| 245 | 4933.64 | 20.57 |
| 246 | 4986.98 | 21.35 |
| 247 | 5039.63 | 25.68 |
| 248 | 5042.1 | 24.31 |
| 249 | 5060.4 | 20.09 |
| 250 | 5110.36 | 25.28 |
| 251 | 5122.44 | 20.75 |
| 252 | 5157.79 | 34.26 |
| 253 | 5181.63 | 25.36 |
| 254 | 5258.56 | 22.09 |
| 255 | 5439.46 | 25.41 |
| 256 | 5495.33 | 31.72 |
| 257 | 5660.6 | 31.55 |
| 258 | 5745.2 | 19.72 |
| 259 | 5891.27 | 24.16 |
| 260 | 6224.17 | 25.27 |
| 261 | 6327.46 | 22.03 |
| 262 | 6498.75 | 20.1 |
| 263 | 6611.97 | 28.44 |
| 264 | 6617.76 | 24.03 |
| 265 | 6813.98 | 23.14 |
| 266 | 6881.81 | 21.72 |
| 267 | 6893.8 | 24.57 |
| 268 | 6985.31 | 21.87 |
| 269 | 7026.99 | 22.02 |
| 270 | 7049.04 | 22.79 |
| 271 | 7106.83 | 23 |
| 272 | 7142.49 | 21.32 |
| 273 | 7210.86 | 22.94 |
| 274 | 8176.16 | 19.47 |
| 275 | 8186.93 | 20.72 |
| 276 | 8289.28 | 19.39 |
| 277 | 9724.01 | 25.64 |
| 278 | 12529 | 20.64 |
| 279 | 14557.83 | 19.21 |

### BRIEF DESCRIPTION OF THE DRAWINGS

FIG. 1a is a graph plotting sensitivity vs. 100-specificity for biomarkers with the IDs 108317 (ID paper 356), 108983 (ID paper 362), ID 128206 (ID paper 472), ID 131316 (ID paper 490), ID 131401 (ID paper 491) and ID 136537 (ID paper 515);

FIG. 1b graph plotting sensitivity vs. 100-specificity for biomarkers with the IDs 49693 (ID paper 51), 66564 (ID paper 111), ID 75674 (ID paper 142), ID 89174 (ID paper 208);

FIGS. 2a and b are graphs plotting sensitivity vs. 100-specificity for twelve markers according to the present disclosure.

### DETAILED DESCRIPTION OF THE PREFERRED EMBODIMENT

With the present disclosure, it is possible to diagnose Alzheimer's disease very early and reliably. Thus, the disease can be cured at an early stage. The disclosure further enables an inexpensive, quick and reliable diagnosis with only minimalinvasive operations.

The migration time is determined by capillary electrophoresis (CE), for example, as set forth in the Example under item 2. In this Example, a glass capillary of 90 cm in length and with an inner diameter (ID) of 50 μm and an outer diameter (OD) of 360 μm is operated at an applied voltage of 30 kV. As the mobile solvent, 300% methanol, 0.50% formic acid in water is used.

It is known that the CE migration times may vary. Nevertheless, the order in which the polypeptide markers are eluted is typically the same under the stated conditions for any CE system employed. In order to balance any differences in the migration time that may nevertheless occur, the system can be normalized using standards for which the migration times are exactly known. These standards may be, for example, the polypeptides stated in the Examples (see the Example, item 3).

The characterization of the polypeptides shown in Tables 1 to 3 was determined by means of capillary electrophoresis-mass spectrometry (CE-MS), a method which has been described in detail, for example, by Neuhoff et al. (Rapid communications in mass spectrometry, 2004, Vol. 20, pages 149-156). The variation of the molecular masses between individual measurements or between different mass spectrometers is relatively small when the calibration is exact, typically within a range of ±0.10%, preferably within a range of ±0.050%.

The polypeptide markers according to the disclosure are proteins or peptides or degradation products of proteins or peptides. They may be chemically modified, for example, by posttranslational modifications, such as glycosylation, phosphorylation, alkylation or disulfide bridges, or by other reactions, for example, within the scope of degradation. In addition, the polypeptide markers may also be chemically altered, for example, oxidized, during the purification of the samples.

Proceeding from the parameters that determine the polypeptide markers (molecular weight and migration time), it is possible to identify the sequence of the corresponding polypeptides by methods known in the prior art.

The polypeptides according to the disclosure (see Tables 1 to 3) are used to diagnose Alzheimer's disease. “Diagnosis” means the process of knowledge gaining by assigning symptoms or phenomena to a disease or injury. In the present case, the existence of Alzheimer's disease is concluded from the presence or absence of particular polypeptide markers. Thus, the polypeptide markers according to the disclosure are determined in a sample from a subject, wherein its presence or absence allows to conclude the existence of Alzheimer's disease. The presence or absence of a polypeptide marker can be measured by any method known in the prior art. Methods which may be used are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or defined from experience.

In the context of the present disclosure, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of Alzheimer's disease. Table 2 shows polypeptide markers which are typically present in patients with Alzheimer's disease (ill), such as polypeptide markers No. 1 to 50, but absent or rarely present in subjects with no Alzheimer's disease (control). In addition, there are polypeptide markers which are present in subjects with no Alzheimer's disease, but are less frequently or not at all present in subjects with Alzheimer's disease, for example, polypeptide markers No. 43 or 44.

TABLE 2
Polypeptide markers for the diagnosis of Alzheimer's disease, their
molecular masses and migration times as well as their presence and
absence in the individual groups as frequency per group (1 = 100%,
0 = 0%) in the group of patients suffering from Alzheimer's
disease and the control group (sample processing and measurement
as described in the Example).

| | | | | Frequency per group | |
|---|---|---|---|---|---|
| | Mass | Migration | Frequency | Alzheimer's | |
| No. | [g/mol] | time [min] | difference | dementia | Controls |
| 1 | 1073.35 | 34.96 | 0.45 | 0.45 | 0 |
| 2 | 1073.35 | 35.9 | −0.48 | 0.46 | 0.94 |
| 3 | 1110.31 | 37.67 | −0.44 | 0 | 0.44 |
| 4 | 1111.3 | 34.97 | −0.41 | 0.27 | 0.69 |
| 5 | 1196.36 | 36.37 | −0.44 | 0.31 | 0.75 |
| 6 | 1265.63 | 27.22 | 0.41 | 0.66 | 0.25 |
| 7 | 1322.44 | 36.2 | 0.44 | 0.75 | 0.31 |
| 8 | 1387.07 | 20.42 | −0.41 | 0.09 | 0.5 |
| 9 | 1390.49 | 36.26 | 0.42 | 0.55 | 0.13 |
| 10 | 1423.57 | 29.85 | 0.48 | 0.79 | 0.31 |
| 11 | 1453.55 | 30.47 | 0.44 | 0.63 | 0.19 |
| 12 | 1490.43 | 35.48 | 0.42 | 0.79 | 0.38 |
| 13 | 1494.62 | 30.23 | 0.5 | 0.75 | 0.25 |
| 14 | 1819.86 | 23.51 | −0.48 | 0.27 | 0.75 |
| 15 | 1927.02 | 21 | 0.48 | 0.67 | 0.19 |
| 16 | 2128.97 | 26.03 | 0.46 | 0.96 | 0.5 |
| 17 | 2214.22 | 26.48 | 0.52 | 0.83 | 0.31 |
| 18 | 2313.26 | 30.32 | 0.48 | 0.67 | 0.19 |
| 19 | 2423.19 | 27.03 | 0.41 | 0.6 | 0.19 |
| 20 | 2428.11 | 27.16 | −0.45 | 0.36 | 0.81 |
| 21 | 2521.33 | 27.86 | −0.44 | 0 | 0.44 |
| 22 | 2644.31 | 21.1 | 0.45 | 0.89 | 0.44 |
| 23 | 2706.37 | 28.3 | 0.46 | 0.71 | 0.25 |
| 24 | 3068.46 | 30.03 | 0.46 | 0.77 | 0.31 |
| 25 | 3079.61 | 37.02 | −0.42 | 0.46 | 0.88 |
| 26 | 3153.38 | 35.65 | 0.46 | 0.71 | 0.25 |
| 27 | 3229.79 | 25.02 | −0.44 | 0 | 0.44 |
| 28 | 3438.77 | 23.49 | 0.44 | 0.75 | 0.31 |
| 29 | 3481.8 | 30.62 | 0.45 | 0.89 | 0.44 |
| 30 | 3567.61 | 23.88 | −0.44 | 0.18 | 0.63 |
| 31 | 3685.85 | 25.47 | −0.44 | 0.18 | 0.63 |
| 32 | 3921.84 | 29.49 | −0.41 | 0.09 | 0.5 |
| 33 | 4036.3 | 24.92 | 0.5 | 0.69 | 0.19 |
| 34 | 4038.22 | 20.53 | −0.63 | 0 | 0.63 |
| 35 | 4093.96 | 21.04 | 0.5 | 0.63 | 0.13 |
| 36 | 4145.22 | 24.51 | 0.42 | 0.42 | 0 |
| 37 | 4372.3 | 19.37 | −0.44 | 0.18 | 0.63 |
| 38 | 4377.95 | 30.81 | 0.5 | 0.63 | 0.13 |
| 39 | 4496.98 | 19.35 | −0.41 | 0.09 | 0.5 |
| 40 | 4516 | 19.37 | −0.45 | 0.36 | 0.81 |
| 41 | 4540.4 | 22 | −0.41 | 0.09 | 0.5 |
| 42 | 4586.98 | 19.38 | −0.41 | 0.27 | 0.69 |
| 43 | 4596.23 | 19.6 | −0.47 | 0.09 | 0.56 |
| 44 | 4810.95 | 19.77 | −0.44 | 0 | 0.44 |
| 45 | 5227.91 | 25.08 | 0.54 | 0.73 | 0.19 |
| 46 | 5582.16 | 24.3 | 0.4 | 0.4 | 0 |
| 47 | 6402.73 | 22.3 | 0.44 | 0.63 | 0.19 |
| 48 | 7749.32 | 19.97 | −0.41 | 0.09 | 0.5 |
| 49 | 7760.02 | 28.72 | 0.46 | 0.71 | 0.25 |
| 50 | 10671.25 | 28.92 | 0.48 | 0.67 | 0.19 |

In addition or also alternatively to the frequency markers (determination of presence or absence), the amplitude markers as stated in Table 3 may also be used for the diagnosis of Alzheimer's disease (Nos. 51-279). Amplitude markers are used in such a way that the presence or absence is not critical, but the height of the signal (the amplitude) decides if the signal is present in both groups. In Table 3, the mean amplitudes of the corresponding signals (characterized by mass and migration time) averaged over all samples measured are stated. To achieve comparability between differently concentrated samples or different measuring methods, all peptide signals of a sample are normalized to a total amplitude of 1 million counts. Therefore, the respective mean amplitudes of the individual markers are stated as parts per million (ppm). All groups employed consist of at least 15 individual patient or control samples in order to obtain a reliable mean amplitude. The decision for a diagnosis (Alzheimer's or not) is made as a function of how high the amplitude of the respective polypeptide markers in the patient sample is in comparison with the mean amplitudes in the control groups or the Alzheimer group. If the mean amplitudes rather correspond to the mean amplitudes of the Alzheimer group, the existence of Alzheimer's disease is to be considered, and if it rather corresponds to the mean amplitudes of the control group, the non-existence of Alzheimer's disease is to be considered. A more exact definition shall be given by means of marker No. 130 (Table 3). The mean amplitude of the marker is significantly increased in Alzheimer's disease (920 ppm vs. 373 ppm in the control group). Now, if the value for this marker in a patient sample is from 0 to 373 ppm or exceeds this range by a maximum of 20%, i.e., from 0 to 448 ppm, then this sample belongs to the control group. If the value is around 920 ppm or up to 20% below, or higher, i.e., between 736 and very high values, the existence of Alzheimer's disease is to be considered. The smaller the distance between the amplitudes of the control group and the Alzheimer group, the closer a value that lies between the two reference values has to be to one of the reference values.

One possibility is to subdivide the range between the mean amplitudes into three portions. If the value is in the lower third, this is indicative of the lower value; if the value is in the upper third, this is indicative of the upper value. If it is in the middle third, a definite statement about this marker is not possible.

TABLE 3
Amplitude markers

| | | |
|---|---|---|
| Mass | Migration | mean amplitude per group |

| No. | [g/mol] | time [min] | Alzheimer | Controls |
|---|---|---|---|---|
| 51 | 824.48 | 19.92 | 568.85 | 501.97 |
| 52 | 840.49 | 19.92 | 810.27 | 593.64 |
| 53 | 846.38 | 25.96 | 100.62 | 122.25 |
| 54 | 897.41 | 25.86 | 81.54 | 48.26 |
| 55 | 911.3 | 34.1 | 242.62 | 247.96 |
| 56 | 950.57 | 24.13 | 78.07 | 67.87 |
| 57 | 973.26 | 35.47 | 179.96 | 150.04 |
| 58 | 984.45 | 26.32 | 110.69 | 132.44 |
| 59 | 988.57 | 35.65 | 211.75 | 188.31 |
| 60 | 1013.43 | 25.18 | 175.63 | 104.85 |
| 61 | 1016.3 | 35.53 | 909.17 | 810.15 |
| 62 | 1041.58 | 36.79 | 86.97 | 139.71 |
| 63 | 1053.52 | 25.25 | 87.43 | 46.52 |
| 64 | 1055.61 | 25.7 | 277.17 | 465.99 |
| 65 | 1082.55 | 23.75 | 78.89 | 50.53 |
| 66 | 1082.65 | 26.15 | 112.48 | 104.55 |
| 67 | 1083.5 | 27.14 | 118.51 | 108.08 |
| 68 | 1083.55 | 25.61 | 271.44 | 164.33 |
| 69 | 1085.63 | 36.39 | 140.8 | 145.98 |
| 70 | 1094.61 | 25.99 | 460.11 | 418.75 |
| 71 | 1130.37 | 35.19 | 670.41 | 372.37 |
| 72 | 1130.63 | 27.07 | 115.03 | 78.26 |
| 73 | 1135.56 | 27.03 | 60.46 | 58.13 |
| 74 | 1150.61 | 26.55 | 80.41 | 45.34 |
| 75 | 1158.62 | 27.02 | 68.79 | 40.98 |
| 76 | 1183.66 | 27.49 | 110.3 | 73.65 |
| 77 | 1186.56 | 27.21 | 315.86 | 221.73 |
| 78 | 1217.56 | 21.8 | 90.54 | 80.33 |
| 79 | 1229.62 | 27.38 | 99.75 | 62.81 |
| 80 | 1250.61 | 27.59 | 455.17 | 151.8 |
| 81 | 1276.43 | 35.7 | 1739.68 | 1000.12 |
| 82 | 1283.4 | 35.91 | 104.76 | 53 |
| 83 | 1300.67 | 29.7 | 120.17 | 58.65 |
| 84 | 1306.74 | 22.16 | 552.66 | 320.23 |
| 85 | 1333.44 | 35.93 | 268.17 | 204.63 |
| 86 | 1342.44 | 35.43 | 275.61 | 325.49 |
| 87 | 1347.71 | 28.94 | 83.29 | 67.88 |
| 88 | 1349.67 | 28.25 | 363.22 | 580.39 |
| 89 | 1360.67 | 28.06 | 90.7 | 94.56 |
| 90 | 1386.68 | 28.62 | 366.31 | 404.69 |
| 91 | 1388.74 | 27.97 | 319.52 | 394.09 |
| 92 | 1399.44 | 36.06 | 179.15 | 82.16 |
| 93 | 1404.73 | 29.49 | 165.77 | 108.43 |
| 94 | 1409.63 | 22.11 | 288.37 | 317.52 |
| 95 | 1449.7 | 22.04 | 77.35 | 102.04 |
| 96 | 1451.72 | 28.84 | 111.94 | 52.91 |
| 97 | 1464.7 | 30.57 | 1169.16 | 1453.9 |
| 98 | 1464.7 | 30.57 | 1346.04 | 1453.9 |
| 99 | 1475.77 | 30.05 | 216.71 | 146.11 |
| 100 | 1499.74 | 29.89 | 104.53 | 57.36 |
| 101 | 1519.76 | 31.87 | 392.54 | 271.33 |
| 102 | 1535.74 | 30 | 2366.44 | 3661.09 |
| 103 | 1542.78 | 23.24 | 713.91 | 533.83 |
| 104 | 1550.67 | 27.47 | 576.53 | 637.58 |
| 105 | 1552.61 | 30.78 | 176.95 | 110.5 |
| 106 | 1590.81 | 32.46 | 1491.15 | 1260.68 |
| 107 | 1596.83 | 30.32 | 873.36 | 577.23 |
| 108 | 1614.84 | 31.54 | 688.7 | 410.65 |
| 109 | 1623.65 | 31.1 | 697.2 | 364.52 |
| 110 | 1628.8 | 20.75 | 304.52 | 326.72 |
| 111 | 1636.79 | 22.71 | 1006.43 | 814.9 |
| 112 | 1659.81 | 27.41 | 589.62 | 358.41 |
| 113 | 1692.91 | 31.38 | 127.4 | 95.57 |
| 114 | 1703.89 | 33.01 | 287.84 | 258.89 |
| 115 | 1727.92 | 32.13 | 552.58 | 276.12 |
| 116 | 1732.88 | 31.36 | 596.19 | 543.08 |
| 117 | 1738.68 | 32.16 | 92.84 | 40.42 |
| 118 | 1741.83 | 30.56 | 114.01 | 84.13 |
| 119 | 1756.89 | 19.81 | 176.68 | 85.89 |
| 120 | 1771.97 | 30.02 | 328.73 | 214.56 |
| 121 | 1800.02 | 25 | 516.9 | 674.37 |
| 122 | 1816.98 | 33.53 | 395.82 | 180.33 |
| 123 | 1820.06 | 32.23 | 107.09 | 76.57 |
| 124 | 1841.77 | 36.26 | 847.46 | 1396.48 |
| 125 | 1849.68 | 18.31 | 405.84 | 688.91 |
| 126 | 1850.96 | 31.97 | 116.29 | 133.34 |
| 127 | 1867.72 | 33.07 | 1356.26 | 956.64 |
| 128 | 1869.92 | 31.28 | 223.93 | 140.79 |
| 129 | 1873 | 21.26 | 440.7 | 270.85 |
| 130 | 1900.07 | 24.19 | 920.26 | 373.01 |
| 131 | 1914.07 | 25.67 | 1519.39 | 1746.72 |
| 132 | 1915.72 | 34.24 | 180.37 | 151.24 |
| 133 | 1917.03 | 24.99 | 197.09 | 216.02 |
| 134 | 1955.95 | 27.81 | 129.24 | 99.09 |
| 135 | 2021.11 | 21.85 | 346.14 | 257.95 |
| 136 | 2041.99 | 32.56 | 193.08 | 127.84 |
| 137 | 2053.18 | 20.54 | 501.38 | 674.91 |
| 138 | 2072.15 | 27.04 | 601.86 | 494.89 |
| 139 | 2077.07 | 25.44 | 261.1 | 214.6 |
| 140 | 2085.12 | 33.75 | 215.62 | 302.81 |
| 141 | 2140.12 | 20.56 | 256.95 | 290.52 |
| 142 | 2166.09 | 27.84 | 128.57 | 52.54 |
| 143 | 2168.66 | 34.88 | 257.1 | 291.77 |
| 144 | 2179.03 | 25.31 | 84.93 | 31.65 |
| 145 | 2278.11 | 26.17 | 438.37 | 314.65 |
| 146 | 2283.18 | 26.31 | 619.59 | 634.52 |
| 147 | 2314.3 | 22.71 | 1018.08 | 1167.69 |
| 148 | 2320.43 | 23.21 | 212.67 | 166.8 |
| 149 | 2327.24 | 34.9 | 1737.42 | 1517.73 |
| 150 | 2341.12 | 26.74 | 304.17 | 535.15 |
| 151 | 2343.24 | 34.95 | 270.79 | 236.16 |
| 152 | 2368.24 | 21.24 | 101.48 | 105.72 |
| 153 | 2377.25 | 28.07 | 2711.11 | 1749.4 |
| 154 | 2390.26 | 27.33 | 617.62 | 500.67 |
| 155 | 2392.73 | 35.48 | 512.31 | 482.03 |
| 156 | 2409.28 | 27.64 | 275.81 | 159.58 |
| 157 | 2410.3 | 30.62 | 338.59 | 232.89 |
| 158 | 2448.39 | 21.07 | 488.24 | 440.74 |
| 159 | 2475.21 | 27.09 | 757.06 | 535.45 |
| 160 | 2475.27 | 24.74 | 478.15 | 297.04 |
| 161 | 2486.04 | 35.76 | 2280.47 | 1824.17 |
| 162 | 2500.33 | 19.99 | 110.01 | 94.92 |
| 163 | 2521.36 | 19.26 | 516.08 | 635.2 |
| 164 | 2522.35 | 27.9 | 4135.44 | 3306.09 |
| 165 | 2527.42 | 19.79 | 393.22 | 500.27 |
| 166 | 2549.29 | 27.66 | 99.25 | 108.48 |
| 167 | 2584.37 | 35.6 | 451.18 | 260.07 |
| 168 | 2684.32 | 21.38 | 1926.21 | 155.86 |
| 169 | 2698.41 | 22.52 | 182.49 | 220.4 |
| 170 | 2799.17 | 25.07 | 445.75 | 375.83 |
| 171 | 2816.31 | 28.74 | 1366.84 | 1928.38 |
| 172 | 2851.42 | 27.7 | 495.9 | 288.67 |
| 173 | 2923.51 | 36.73 | 818.47 | 883.22 |
| 174 | 2982.72 | 19.97 | 3750.78 | 5523.72 |
| 175 | 3019.54 | 24.72 | 170.57 | 189.58 |
| 176 | 3173.68 | 22.95 | 155.36 | 252.28 |
| 177 | 3192.67 | 37.66 | 689.53 | 1089.16 |
| 178 | 3215.61 | 28.53 | 1305.23 | 2016.28 |
| 179 | 3218.4 | 30.31 | 1384.58 | 1875.79 |
| 180 | 3221.59 | 22.7 | 170.45 | 217.24 |
| 181 | 3279.66 | 25.32 | 141.5 | 83 |
| 182 | 3282.34 | 36.05 | 1082.36 | 1351.09 |
| 183 | 3298.42 | 36.17 | 268.81 | 282.69 |
| 184 | 3302.68 | 23.31 | 316.81 | 508.67 |
| 185 | 3324.7 | 21.62 | 422.39 | 490.7 |
| 186 | 3386.77 | 22.21 | 670.34 | 395.98 |
| 187 | 3401.73 | 23.48 | 2618.95 | 4622.56 |
| 188 | 3408.76 | 26.04 | 175.22 | 94.81 |
| 189 | 3462.68 | 21.25 | 2161.32 | 2653.08 |
| 190 | 3466.75 | 23.9 | 904.51 | 559.21 |
| 191 | 3524.8 | 31.72 | 1130.3 | 410.29 |
| 192 | 3529.84 | 23.7 | 1979.26 | 2916.13 |
| 193 | 3595.77 | 23.89 | 344.53 | 455.24 |
| 194 | 3605.4 | 21.53 | 321.29 | 479.63 |
| 195 | 3614.82 | 22.01 | 161.46 | 267.85 |
| 196 | 3644.79 | 29.39 | 1038.75 | 749.16 |
| 197 | 3645.25 | 26.69 | 1161.31 | 741.33 |
| 198 | 3650.74 | 19.9 | 1029.39 | 1161.19 |
| 199 | 3672.71 | 21.9 | 1896.48 | 819.91 |
| 200 | 3677.74 | 24.3 | 1717.39 | 1567.09 |
| 201 | 3685.86 | 22.21 | 9259.98 | 16077.47 |
| 202 | 3687.78 | 20.58 | 832.51 | 1095.77 |
| 203 | 3693.93 | 27.15 | 344.49 | 218.63 |
| 204 | 3741.63 | 20.1 | 547.74 | 806.46 |
| 205 | 3768.77 | 31.84 | 533.93 | 377.59 |
| 206 | 3782.94 | 22.68 | 360.79 | 364.04 |
| 207 | 3788.04 | 25.24 | 609.58 | 782.85 |
| 208 | 3792.76 | 27.25 | 682.32 | 432.09 |
| 209 | 3819.82 | 32.74 | 120.7 | 133.76 |
| 210 | 3881.87 | 24.56 | 3257.71 | 1968.09 |
| 211 | 3903.08 | 25.71 | 2177.14 | 2780.86 |
| 212 | 3905.8 | 27.72 | 23257.92 | 16304.43 |
| 213 | 3929.73 | 23.07 | 2742.02 | 3430.19 |
| 214 | 3934.76 | 24.02 | 653.19 | 487.54 |
| 215 | 3951.07 | 22.77 | 25509.01 | 32844.44 |
| 216 | 4005.54 | 22.57 | 566.89 | 387.06 |
| 217 | 4087.12 | 26.92 | 607.44 | 490.16 |
| 218 | 4112.78 | 24.46 | 267.94 | 308.15 |
| 219 | 4131.68 | 22.54 | 653.71 | 586.61 |
| 220 | 4164.86 | 25.11 | 781.59 | 466.12 |
| 221 | 4192.99 | 34.95 | 1761.93 | 1496.61 |
| 222 | 4203.13 | 26.14 | 613.9 | 1051.15 |
| 223 | 4218.31 | 20.6 | 939.4 | 1360.44 |
| 224 | 4232.32 | 32.35 | 318.48 | 349.1 |
| 225 | 4250 | 27.68 | 504.35 | 286.25 |
| 226 | 4278 | 25.3 | 1161.81 | 745.21 |
| 227 | 4301.93 | 21.26 | 476.37 | 698.84 |
| 228 | 4307.09 | 31.58 | 157.37 | 88.2 |
| 229 | 4349.06 | 28.17 | 15449.01 | 7992.31 |
| 230 | 4384.07 | 19.8 | 465.18 | 981.39 |
| 231 | 4428.79 | 32.86 | 827.48 | 433.08 |
| 232 | 4464.64 | 21.54 | 4084.32 | 2311.8 |
| 233 | 4468.61 | 23.47 | 3429.99 | 2945.88 |
| 234 | 4510.97 | 28.56 | 1157.68 | 629.08 |
| 235 | 4548.03 | 25.95 | 3177.96 | 2703.21 |
| 236 | 4562 | 29.31 | 509.94 | 403.2 |
| 237 | 4562 | 29.31 | 574.68 | 403.2 |
| 238 | 4564.18 | 26.01 | 497.67 | 438.34 |
| 239 | 4583.84 | 24.2 | 7608.37 | 7241.51 |
| 240 | 4637.81 | 27.82 | 337.27 | 294.89 |
| 241 | 4730.71 | 19.63 | 797.93 | 1759.4 |
| 242 | 4749.41 | 21.81 | 725.74 | 426.89 |
| 243 | 4750.06 | 19.53 | 4365.88 | 3189.94 |
| 244 | 4890.59 | 23.27 | 1965.12 | 1418.61 |
| 245 | 4933.64 | 20.57 | 878.93 | 735.48 |
| 246 | 4986.98 | 21.35 | 637.73 | 533.05 |
| 247 | 5039.63 | 25.68 | 1039.64 | 1389.17 |
| 248 | 5042.1 | 24.31 | 3686.99 | 3120.74 |
| 249 | 5060.4 | 20.09 | 3307.86 | 1942.65 |
| 250 | 5110.36 | 25.28 | 637.44 | 697.57 |
| 251 | 5122.44 | 20.75 | 1342 | 851.23 |
| 252 | 5157.79 | 34.26 | 200.52 | 208.01 |
| 253 | 5181.63 | 25.36 | 5716.39 | 4305.72 |
| 254 | 5258.56 | 22.09 | 12476.68 | 10602.4 |
| 255 | 5439.46 | 25.41 | 713.92 | 522.3 |
| 256 | 5495.33 | 31.72 | 1776.93 | 1297.43 |
| 257 | 5660.6 | 31.55 | 893.02 | 595.49 |
| 258 | 5745.2 | 19.72 | 851.4 | 1608.76 |
| 259 | 5891.27 | 24.16 | 482.2 | 422.63 |
| 260 | 6224.17 | 25.27 | 1104.36 | 995.62 |
| 261 | 6327.46 | 22.03 | 1410.46 | 1027.79 |
| 262 | 6498.75 | 20.1 | 3989.56 | 2972.62 |
| 263 | 6611.97 | 28.44 | 311.21 | 338.55 |
| 264 | 6617.76 | 24.03 | 2799.82 | 1943.58 |
| 265 | 6813.98 | 23.14 | 4217.01 | 3699.9 |
| 266 | 6881.81 | 21.72 | 450.1 | 868.21 |
| 267 | 6893.8 | 24.57 | 2537.57 | 1569.65 |
| 268 | 6985.31 | 21.87 | 2238.63 | 1577.04 |
| 269 | 7026.99 | 22.02 | 2132.66 | 1480.92 |
| 270 | 7049.04 | 22.79 | 8522.63 | 6229.31 |
| 271 | 7106.83 | 23 | 766.91 | 575.59 |
| 272 | 7142.49 | 21.32 | 1014.86 | 557.95 |
| 273 | 7210.86 | 22.94 | 915.21 | 568.24 |
| 274 | 8176.16 | 19.47 | 11495.92 | 7835.63 |
| 275 | 8186.93 | 20.72 | 1706.07 | 1130.11 |
| 276 | 8289.28 | 19.39 | 4317.47 | 3227.21 |
| 277 | 9724.01 | 25.64 | 3380.24 | 1957.82 |
| 278 | 12529 | 20.64 | 38234.95 | 9397.12 |
| 279 | 14557.83 | 19.21 | 1514.79 | 2225.19 |

The subject from which the sample in which the presence or absence of one or more polypeptide markers is determined is derived may be any subject which is capable of suffering from Alzheimer's disease, for example, an animal or human. Preferably, the subject is a mammal, such as a dog or a horse, and most preferably, it is a human.

In a preferred embodiment of the disclosure, not just one polypeptide marker, but a combination of markers are used to diagnose Alzheimer's disease, wherein the existence of Alzheimer's disease is concluded from their presence or absence. By comparing a plurality of polypeptide markers, a bias in the overall result from a few individual deviations from the typical presence probability in the sick or control individual can be reduced or avoided.

The sample in which the presence or absence of the polypeptide marker or markers according to the disclosure is measured may be any sample which is obtained from the body of the subject. The sample is a sample which has a polypeptide composition suitable for providing information about the state of the subject (Alzheimer's disease or not). For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample.

In a preferred embodiment, the sample is a urine sample, blood sample, wherein said blood sample may be a (blood) serum or (blood) plasma sample, or a cerebrospinal fluid sample. The cerebrospinal fluid is a liquid that is in contact with the brain and also bathes the spinal cord. From the spinal cord, it can also be withdrawn by puncture with a low expenditure.

Blood samples can be taken by methods known in the prior art, for example, from a vein, artery or capillary. Usually, a blood sample is obtained by withdrawing venous blood by means of a syringe, for example, from an arm of the subject. The term “blood sample” includes samples obtained from blood by further purification and separation methods known from the prior art, such as blood plasma or blood serum.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine or blood sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, or electrophoretic separation. Particular examples thereof are gel electrophoresis, two-dimensional polyacrylamide gel electrophoresis (2D-PAGE), capillary electrophoresis, metal affinity chromatography, immobilized metal affinity chromatography (IMAC), lectin-based affinity chromatography, liquid chromatography, highperformance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the disclosure, the sample, before being measured, is separated by capillary electrophoresis, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kDa protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas the matrix-assisted laser desorption/ionization (MALDI) technique is used for measuring ions from a sample crystallized with a matrix. For analyzing the ions formed, quadrupoles, ion traps or time-of-flight (TOF) analyzers may be used.

In electrospray ionization (ESI), the molecules present in solution are atomized, inter alia, under the influence of high voltage (e.g., 1-8 kV), which forms charged droplets that become smaller from the evaporation of the solvent. Finally, so-called Coulomb explosions cause the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a very high resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC-MS (liquid chromatography/mass spectrometry), 2D-PAGE/MS and capillary electrophoresis-mass spectrometry (CE-MS). All methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J Chromatogr A, 2003, Vol. 1013: 157-171, and Electrophoresis, 2004, 25: 2044-2055) and in Wittke et al. (Journal of Chromatography A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared. This pattern can be compared with reference patterns of sick or healthy subjects. In most cases, it is sufficient to use a limited number of polypeptide markers for the diagnosis of Alzheimer's disease. A CE-MS method which includes CE coupled on-line to an ESI-TOF MS device is further preferred.

For CE-MS, the use of volatile solvents is preferred, and it is best to work under essentially salt-free conditions. Examples of suitable solvents include acetonitrile, methanol and the like. The solvents can be diluted with water or admixed with a weak acid (e.g., 0.10% formic acid) in order to protonate the analyte, preferably the polypeptides.

By means of capillary electrophoresis, it is possible to separate molecules by their charge and size. Neutral particles will migrate at the speed of the electroosmotic flow upon application of a current, while cations are accelerated towards the cathode, and anions are delayed. The advantage of capillaries in electrophoresis resides in their favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of from 50 to 75 μm are usually employed. The lengths employed are from 30 to 100 cm. In addition, the capillaries are usually made of plastic-coated silica glass. The capillaries may be both untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the performance or altered meanwhile.

In a preferred method for measuring polypeptide markers, the markers of the sample are separated by means of capillary electrophoresis, then directly ionized and transferred on-line to a mass spectrometer coupled thereto for detection.

In the method according to the disclosure, it is advantageous to use several polypeptide markers for the diagnosis of Alzheimer's disease. In particular, at least three polypeptide markers may be used, for example, markers 1, 2 and 3; 1, 2 and 4; etc.

More preferred is the use of at least 4, 5 or 6 markers.

Even more preferred is the use of at least 13 markers, for example, markers 1 to 13.

Most preferred is the use of all 279 markers listed in Tables 1 to 3.

In one embodiment, markers 123, 144, 167, 38, 255, 257 and 72 are employed.

In order to determine the probability of the existence of Alzheimer's disease when several markers are used, statistic methods known to the skilled person may be used. For example, the Random Forests method described by Weissinger et al. (Kidney Int., 2004, 65: 2426-2434) may be used by using a computer program such as S-Plus.

### EXAMPLE

### 1. Sample Preparation

For detecting the polypeptide markers for Alzheimer's disease, cerebrospinal fluid was employed. Cerebrospinal fluid was withdrawn by a lumbar puncture from healthy donors (control group) as well as from patients suffering from Alzheimer's disease. Control samples from 6 persons (age 32-64 years) without neurological or psychiatric diseases were used. The cerebrospinal fluid samples for the Alzheimer group were derived from 23 patients (age 57-76 years), those for the MCI group were derived from 8 patients (age 60-75 years).

For the subsequent CE-MS measurement, the large proteins occurring in cerebrospinal fluid, such as albumin and immunoglobulins, had to be separated off by ultrafiltration. Thus, 700 μl of cerebrospinal fluid was removed and admixed with 700 μl of filtration buffer (4 M urea, 10 mM NH₄OH, 0.02% SDS). This 1.4 ml of sample volume was ultrafiltrated (Amicon 30 kDa, Millipore, Bedford, USA). The ultrafiltration was performed at 3000 rpm in a centrifuge until 1.2 ml of ultrafiltrate was obtained.

The 1.2 ml of filtrate obtained was then applied to a Pharmacia C-2 column (Pharmacia, Uppsala, Sweden) in order to remove urea, salts and other disturbing components. The bound polypeptides were then eluted from the C-2 column with 500% acetonitrile, 0.50% formic acid in water, and lyophilized. For the CE-MS measurement, the polypeptides were resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 30% methanol and 0.5% formic acid in water. For the “sheath flow” on the MS, 30% isopropanol with 0.5% formic acid was used at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With a pressure of 1 psi, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. After the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good as possible a signal intensity on the side of the MS, the nebulizer gas was set to the lowest possible value. The voltage applied for generating the electrospray was 3700-4100 V. The remaining settings at the mass spectrometer were optimized for peptide detection according to the manufacturer's protocol. The spectra were recorded over a mass range of m/z 350 to m/z 3000 and accumulated every 3 seconds.

### 3. Standards for the CE Measurement

For checking and calibrating the CE measurement, the following proteins or polypeptides which are characterized by the stated CE migration times under the selected conditions were employed:

| Protein/polypeptide | Migration time | |
|---|---|---|
| Aprotinin (SIGMA, Taufkirchen, DE, Cat. # A1153) | 9.2 | min |
| Ribonuclease (SIGMA, Taufkirchen, DE, Cat. # R4875) | 10.9 | min |
| Lysozyme (SIGMA, Taufkirchen, DE, Cat. # L7651) | 8.9 | min |
| “REV”, Sequence: REVQSKIGYGRQIIS | 15.6 | min |
| “ELM”, Sequence: ELMTGELPYSHINNRDQIIFMVGR | 23.4 | min |
| “KINCON”, Sequence: TGSLPYSHIGSRDQIIFMVGR | 20.0 | min |
| “GIVLY” Sequence: GIVLYELMTGELPYSHIN | 36.8 | min |

The proteins/polypeptides were employed at a concentration of 10 pmol/μl each in water. “REV”, “ELM, “KINCON” and “GIVLY” are synthetic peptides.

The molecular masses of the peptides and the m/z ratios of the individual charge states visible in MS are as follows:

| | H (mono) | | | | | | |
|---|---|---|---|---|---|---|---|
| | 1.0079 | 1.0079 | 1.0079 | 1.0079 | 1.0079 | 1.0079 | 1.0079 |
| | Aprotinin | Ribonuclease | Lysozyme | REV | KINCON | ELM | GIVLY |
| | Mono | Mono | Mono | Mono | Mono | Mono | Mono |
| m/z | Mass | Mass | Mass | Mass | Mass | Mass | Mass |
| 0 | 6513.09 | 13681.32 | 14303.88 | 1732.96 | 2333.19 | 2832.41 | 2048.03 |
| 1 | 6514.0979 | 13682.328 | 14304.888 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.559 | 260.2512 | 315.7201 | 228.5668 |
| 10 | 652.3169 | 1369.1399 | 1431.3959 | 174.3039 | 234.3269 | 284.2489 | 205.8109 |
| 11 | 593.107 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

### 4. Comparison of the Markers According to the Disclosure with the Prior Art

In Electrophoresis 26 (2005), 1476-1487, Wittke et al. describe ten markers in Table 1 that are supposed to be indicative of Alzheimer's disease. Further studies have shown that the markers found only have a lower specificity.

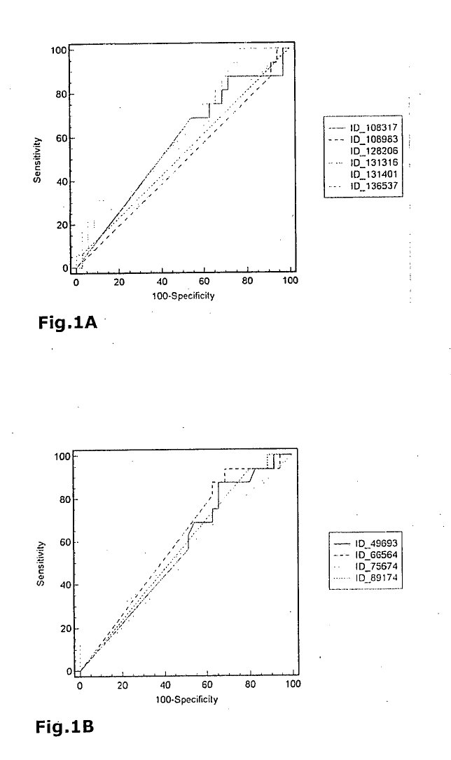

FIG. 1a shows the significance of the biomarkers from this publication. Shown are the biomarkers with the IDs 108317 (ID paper 356), 108983 (ID paper 362), ID 128206 (ID paper 472), ID 131316 (ID paper 490), ID 131401 (ID paper 491) and ID 136537 (ID paper 515).

FIG. 1b shows the significance of further biomarkers from this publication. Shown are the biomarkers with the IDs 49693 (ID paper 51), 66564 (ID paper 111), ID 75674 (ID paper 142), ID 89174 (ID paper 208).

It is clearly seen that the predictive value is almost 0 for almost each of the ten markers included in the publication, in contrast to the markers claimed according to the disclosure.

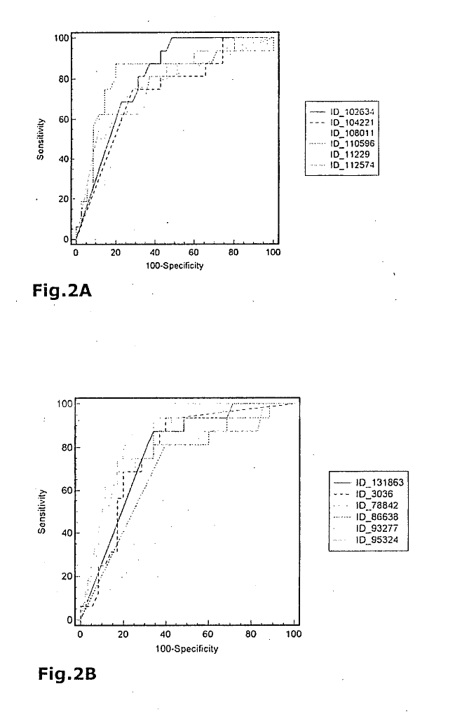

FIGS. 2a and b show the corresponding analysis for twelve markers according to the disclosure. These result in a concrete separation between the groups (healthy vs. Alzheimer's). By selecting at least three markers, the analysis reaches an accuracy of 84%.

1-9. (canceled)

10. A method for the diagnosis of Alzheimer's disease, comprising the step of determining the presence or absence of at least one polypeptide marker in a sample, wherein said polypeptide marker is selected from markers 1 to 50 (frequency markers), or determining the amplitude of at least one polypeptide marker selected from markers 51 to 279 (amplitude markers), which are characterized by the following values for the molecular masses and migration times:

| | | Migration |
|---|---|---|
| | Mass | time |
| No. | [g/mol] | [min] |
| 1 | 1073.35 | 34.96 |
| 2 | 1073.35 | 35.9 |
| 3 | 1110.31 | 37.67 |
| 4 | 1111.3 | 34.97 |
| 5 | 1196.36 | 36.37 |
| 6 | 1265.63 | 27.22 |
| 7 | 1322.44 | 36.2 |
| 8 | 1387.07 | 20.42 |
| 9 | 1390.49 | 36.26 |
| 10 | 1423.57 | 29.85 |
| 11 | 1453.55 | 30.47 |
| 12 | 1490.43 | 35.48 |
| 13 | 1494.62 | 30.23 |
| 14 | 1819.86 | 23.51 |
| 15 | 1927.02 | 21 |
| 16 | 2128.97 | 26.03 |
| 17 | 2214.22 | 26.48 |
| 18 | 2313.26 | 30.32 |
| 19 | 2423.19 | 27.03 |
| 20 | 2428.11 | 27.16 |
| 21 | 2521.33 | 27.86 |
| 22 | 2644.31 | 21.1 |
| 23 | 2706.37 | 28.3 |
| 24 | 3068.46 | 30.03 |
| 25 | 3079.61 | 37.02 |
| 26 | 3153.38 | 35.65 |
| 27 | 3229.79 | 25.02 |
| 28 | 3438.77 | 23.49 |
| 29 | 3481.8 | 30.62 |
| 30 | 3567.61 | 23.88 |
| 31 | 3685.85 | 25.47 |
| 32 | 3921.84 | 29.49 |
| 33 | 4036.3 | 24.92 |
| 34 | 4038.22 | 20.53 |
| 35 | 4093.96 | 21.04 |
| 36 | 4145.22 | 24.51 |
| 37 | 4372.3 | 19.37 |
| 38 | 4377.95 | 30.81 |
| 39 | 4496.98 | 19.35 |
| 40 | 4516 | 19.37 |
| 41 | 4540.4 | 22 |
| 42 | 4586.98 | 19.38 |
| 43 | 4596.23 | 19.6 |
| 44 | 4810.95 | 19.77 |
| 45 | 5227.91 | 25.08 |
| 46 | 5582.16 | 24.3 |
| 47 | 6402.73 | 22.3 |
| 48 | 7749.32 | 19.97 |
| 49 | 7760.02 | 28.72 |
| 50 | 10671.25 | 28.92 |
| 51 | 824.48 | 19.92 |
| 52 | 840.49 | 19.92 |
| 53 | 846.38 | 25.96 |
| 54 | 897.41 | 25.86 |
| 55 | 911.3 | 34.1 |
| 56 | 950.57 | 24.13 |
| 57 | 973.26 | 35.47 |
| 58 | 984.45 | 26.32 |
| 59 | 988.57 | 35.65 |
| 60 | 1013.43 | 25.18 |
| 61 | 1016.3 | 35.53 |
| 62 | 1041.58 | 36.79 |
| 63 | 1053.52 | 25.25 |
| 64 | 1055.61 | 25.7 |
| 65 | 1082.55 | 23.75 |
| 66 | 1082.65 | 26.15 |
| 67 | 1083.5 | 27.14 |
| 68 | 1083.55 | 25.61 |
| 69 | 1085.63 | 36.39 |
| 70 | 1094.61 | 25.99 |
| 71 | 1130.37 | 35.19 |
| 72 | 1130.63 | 27.07 |
| 73 | 1135.56 | 27.03 |
| 74 | 1150.61 | 26.55 |
| 75 | 1158.62 | 27.02 |
| 76 | 1183.66 | 27.49 |
| 77 | 1186.56 | 27.21 |
| 78 | 1217.56 | 21.8 |
| 79 | 1229.62 | 27.38 |
| 80 | 1250.61 | 27.59 |
| 81 | 1276.43 | 35.7 |
| 82 | 1283.4 | 35.91 |
| 83 | 1300.67 | 29.7 |
| 84 | 1306.74 | 22.16 |
| 85 | 1333.44 | 35.93 |
| 86 | 1342.44 | 35.43 |
| 87 | 1347.71 | 28.94 |
| 88 | 1349.67 | 28.25 |
| 89 | 1360.67 | 28.06 |
| 90 | 1386.68 | 28.62 |
| 91 | 1388.74 | 27.97 |
| 92 | 1399.44 | 36.06 |
| 93 | 1404.73 | 29.49 |
| 94 | 1409.63 | 22.11 |
| 95 | 1449.7 | 22.04 |
| 96 | 1451.72 | 28.84 |
| 97 | 1464.7 | 30.57 |
| 99 | 1475.77 | 30.05 |
| 100 | 1499.74 | 29.89 |
| 101 | 1519.76 | 31.87 |
| 102 | 1535.74 | 30 |
| 103 | 1542.78 | 23.24 |
| 104 | 1550.67 | 27.47 |
| 105 | 1552.61 | 30.78 |
| 106 | 1590.81 | 32.46 |
| 107 | 1596.83 | 30.32 |
| 108 | 1614.84 | 31.54 |
| 109 | 1623.65 | 31.1 |
| 110 | 1628.8 | 20.75 |
| 111 | 1636.79 | 22.71 |
| 112 | 1659.81 | 27.41 |
| 113 | 1692.91 | 31.38 |
| 114 | 1703.89 | 33.01 |
| 115 | 1727.92 | 32.13 |
| 116 | 1732.88 | 31.36 |
| 117 | 1738.68 | 32.16 |
| 118 | 1741.83 | 30.56 |
| 119 | 1756.89 | 19.81 |
| 120 | 1771.97 | 30.02 |
| 121 | 1800.02 | 25 |
| 122 | 1816.98 | 33.53 |
| 123 | 1820.06 | 32.23 |
| 124 | 1841.77 | 36.26 |
| 125 | 1849.68 | 18.31 |
| 126 | 1850.96 | 31.97 |
| 127 | 1867.72 | 33.07 |
| 128 | 1869.92 | 31.28 |
| 129 | 1873 | 21.26 |
| 130 | 1900.07 | 24.19 |
| 131 | 1914.07 | 25.67 |
| 132 | 1915.72 | 34.24 |
| 133 | 1917.03 | 24.99 |
| 134 | 1955.95 | 27.81 |
| 135 | 2021.11 | 21.85 |
| 136 | 2041.99 | 32.56 |
| 137 | 2053.18 | 20.54 |
| 138 | 2072.15 | 27.04 |
| 139 | 2077.07 | 25.44 |
| 140 | 2085.12 | 33.75 |
| 141 | 2140.12 | 20.56 |
| 142 | 2166.09 | 27.84 |
| 143 | 2168.66 | 34.88 |
| 144 | 2179.03 | 25.31 |
| 145 | 2278.11 | 26.17 |
| 146 | 2283.18 | 26.31 |
| 147 | 2314.3 | 22.71 |
| 148 | 2320.43 | 23.21 |
| 149 | 2327.24 | 34.9 |
| 150 | 2341.12 | 26.74 |
| 151 | 2343.24 | 34.95 |
| 152 | 2368.24 | 21.24 |
| 153 | 2377.25 | 28.07 |
| 154 | 2390.26 | 27.33 |
| 155 | 2392.73 | 35.48 |
| 156 | 2409.28 | 27.64 |
| 157 | 2410.3 | 30.62 |
| 158 | 2448.39 | 21.07 |
| 159 | 2475.21 | 27.09 |
| 160 | 2475.27 | 24.74 |
| 161 | 2486.04 | 35.76 |
| 162 | 2500.33 | 19.99 |
| 163 | 2521.36 | 19.26 |
| 164 | 2522.35 | 27.9 |
| 165 | 2527.42 | 19.79 |
| 166 | 2549.29 | 27.66 |
| 167 | 2584.37 | 35.6 |
| 168 | 2684.32 | 21.38 |
| 169 | 2698.41 | 22.52 |
| 170 | 2799.17 | 25.07 |
| 171 | 2816.31 | 28.74 |
| 172 | 2851.42 | 27.7 |
| 173 | 2923.51 | 36.73 |
| 174 | 2982.72 | 19.97 |
| 175 | 3019.54 | 24.72 |
| 176 | 3173.68 | 22.95 |
| 177 | 3192.67 | 37.66 |
| 178 | 3215.61 | 28.53 |
| 179 | 3218.4 | 30.31 |
| 180 | 3221.59 | 22.7 |
| 181 | 3279.66 | 25.32 |
| 182 | 3282.34 | 36.05 |
| 183 | 3298.42 | 36.17 |
| 184 | 3302.68 | 23.31 |
| 185 | 3324.7 | 21.62 |
| 186 | 3386.77 | 22.21 |
| 187 | 3401.73 | 23.48 |
| 188 | 3408.76 | 26.04 |
| 189 | 3462.68 | 21.25 |
| 190 | 3466.75 | 23.9 |
| 191 | 3524.8 | 31.72 |
| 192 | 3529.84 | 23.7 |
| 193 | 3595.77 | 23.89 |
| 194 | 3605.4 | 21.53 |
| 195 | 3614.82 | 22.01 |
| 196 | 3644.79 | 29.39 |
| 197 | 3645.25 | 26.69 |
| 198 | 3650.74 | 19.9 |
| 199 | 3672.71 | 21.9 |
| 200 | 3677.74 | 24.3 |
| 201 | 3685.86 | 22.21 |
| 202 | 3687.78 | 20.58 |
| 203 | 3693.93 | 27.15 |
| 204 | 3741.63 | 20.1 |
| 205 | 3768.77 | 31.84 |
| 206 | 3782.94 | 22.68 |
| 207 | 3788.04 | 25.24 |
| 208 | 3792.76 | 27.25 |
| 209 | 3819.82 | 32.74 |
| 210 | 3881.87 | 24.56 |
| 211 | 3903.08 | 25.71 |
| 212 | 3905.8 | 27.72 |
| 213 | 3929.73 | 23.07 |
| 214 | 3934.76 | 24.02 |
| 215 | 3951.07 | 22.77 |
| 216 | 4005.54 | 22.57 |
| 217 | 4087.12 | 26.92 |
| 218 | 4112.78 | 24.46 |
| 219 | 4131.68 | 22.54 |
| 220 | 4164.86 | 25.11 |
| 221 | 4192.99 | 34.95 |
| 222 | 4203.13 | 26.14 |
| 223 | 4218.31 | 20.6 |
| 224 | 4232.32 | 32.35 |
| 225 | 4250 | 27.68 |
| 226 | 4278 | 25.3 |
| 227 | 4301.93 | 21.26 |
| 228 | 4307.09 | 31.58 |
| 229 | 4349.06 | 28.17 |
| 230 | 4384.07 | 19.8 |
| 231 | 4428.79 | 32.86 |
| 232 | 4464.64 | 21.54 |
| 233 | 4468.61 | 23.47 |
| 234 | 4510.97 | 28.56 |
| 235 | 4548.03 | 25.95 |
| 236 | 4562 | 29.31 |
| 237 | 4562 | 29.31 |
| 238 | 4564.18 | 26.01 |
| 239 | 4583.84 | 24.2 |
| 240 | 4637.81 | 27.82 |
| 241 | 4730.71 | 19.63 |
| 242 | 4749.41 | 21.81 |
| 243 | 4750.06 | 19.53 |
| 244 | 4890.59 | 23.27 |
| 245 | 4933.64 | 20.57 |
| 246 | 4986.98 | 21.35 |
| 247 | 5039.63 | 25.68 |
| 248 | 5042.1 | 24.31 |
| 249 | 5060.4 | 20.09 |
| 250 | 5110.36 | 25.28 |
| 251 | 5122.44 | 20.75 |
| 252 | 5157.79 | 34.26 |
| 253 | 5181.63 | 25.36 |
| 254 | 5258.56 | 22.09 |
| 255 | 5439.46 | 25.41 |
| 256 | 5495.33 | 31.72 |
| 257 | 5660.6 | 31.55 |
| 258 | 5745.2 | 19.72 |
| 259 | 5891.27 | 24.16 |
| 260 | 6224.17 | 25.27 |
| 261 | 6327.46 | 22.03 |
| 262 | 6498.75 | 20.1 |
| 263 | 6611.97 | 28.44 |
| 264 | 6617.76 | 24.03 |
| 265 | 6813.98 | 23.14 |
| 266 | 6881.81 | 21.72 |
| 267 | 6893.8 | 24.57 |
| 268 | 6985.31 | 21.87 |
| 269 | 7026.99 | 22.02 |
| 270 | 7049.04 | 22.79 |
| 271 | 7106.83 | 23 |
| 272 | 7142.49 | 21.32 |
| 273 | 7210.86 | 22.94 |
| 274 | 8176.16 | 19.47 |
| 275 | 8186.93 | 20.72 |
| 276 | 8289.28 | 19.39 |
| 277 | 9724.01 | 25.64 |
| 278 | 12529 | 20.64 |
| 279 | 14557.83 | 19.21 |

11. The method according to claim 10, wherein an evaluation of the determined presence or absence is effected by using the following reference values:

| | | | | Frequency per group | |
|---|---|---|---|---|---|
| | Mass | Migration | Frequency | Alzheimer's | |
| No. | [g/mol] | time [min] | difference | dementia | Controls |
| 1 | 1073.35 | 34.96 | 0.45 | 0.45 | 0 |
| 2 | 1073.35 | 35.9 | −0.48 | 0.46 | 0.94 |
| 3 | 1110.31 | 37.67 | −0.44 | 0 | 0.44 |
| 4 | 1111.3 | 34.97 | −0.41 | 0.27 | 0.69 |
| 5 | 1196.36 | 36.37 | −0.44 | 0.31 | 0.75 |
| 6 | 1265.63 | 27.22 | 0.41 | 0.66 | 0.25 |
| 7 | 1322.44 | 36.2 | 0.44 | 0.75 | 0.31 |
| 8 | 1387.07 | 20.42 | −0.41 | 0.09 | 0.5 |
| 9 | 1390.49 | 36.26 | 0.42 | 0.55 | 0.13 |
| 10 | 1423.57 | 29.85 | 0.48 | 0.79 | 0.31 |
| 11 | 1453.55 | 30.47 | 0.44 | 0.63 | 0.19 |
| 12 | 1490.43 | 35.48 | 0.42 | 0.79 | 0.38 |
| 13 | 1494.62 | 30.23 | 0.5 | 0.75 | 0.25 |
| 14 | 1819.86 | 23.51 | −0.48 | 0.27 | 0.75 |
| 15 | 1927.02 | 21 | 0.48 | 0.67 | 0.19 |
| 16 | 2128.97 | 26.03 | 0.46 | 0.96 | 0.5 |
| 17 | 2214.22 | 26.48 | 0.52 | 0.83 | 0.31 |
| 18 | 2313.26 | 30.32 | 0.48 | 0.67 | 0.19 |
| 19 | 2423.19 | 27.03 | 0.41 | 0.6 | 0.19 |
| 20 | 2428.11 | 27.16 | −0.45 | 0.36 | 0.81 |
| 21 | 2521.33 | 27.86 | −0.44 | 0 | 0.44 |
| 22 | 2644.31 | 21.1 | 0.45 | 0.89 | 0.44 |
| 23 | 2706.37 | 28.3 | 0.46 | 0.71 | 0.25 |
| 24 | 3068.46 | 30.03 | 0.46 | 0.77 | 0.31 |
| 25 | 3079.61 | 37.02 | −0.42 | 0.46 | 0.88 |
| 26 | 3153.38 | 35.65 | 0.46 | 0.71 | 0.25 |
| 27 | 3229.79 | 25.02 | −0.44 | 0 | 0.44 |
| 28 | 3438.77 | 23.49 | 0.44 | 0.75 | 0.31 |
| 29 | 3481.8 | 30.62 | 0.45 | 0.89 | 0.44 |
| 30 | 3567.61 | 23.88 | −0.44 | 0.18 | 0.63 |
| 31 | 3685.85 | 25.47 | −0.44 | 0.18 | 0.63 |
| 32 | 3921.84 | 29.49 | −0.41 | 0.09 | 0.5 |
| 33 | 4036.3 | 24.92 | 0.5 | 0.69 | 0.19 |
| 34 | 4038.22 | 20.53 | −0.63 | 0 | 0.63 |
| 35 | 4093.96 | 21.04 | 0.5 | 0.63 | 0.13 |
| 36 | 4145.22 | 24.51 | 0.42 | 0.42 | 0 |
| 37 | 4372.3 | 19.37 | −0.44 | 0.18 | 0.63 |
| 38 | 4377.95 | 30.81 | 0.5 | 0.63 | 0.13 |
| 39 | 4496.98 | 19.35 | −0.41 | 0.09 | 0.5 |
| 40 | 4516 | 19.37 | −0.45 | 0.36 | 0.81 |
| 41 | 4540.4 | 22 | −0.41 | 0.09 | 0.5 |
| 42 | 4586.98 | 19.38 | −0.41 | 0.27 | 0.69 |
| 43 | 4596.23 | 19.6 | −0.47 | 0.09 | 0.56 |
| 44 | 4810.95 | 19.77 | −0.44 | 0 | 0.44 |
| 45 | 5227.91 | 25.08 | 0.54 | 0.73 | 0.19 |
| 46 | 5582.16 | 24.3 | 0.4 | 0.4 | 0 |
| 47 | 6402.73 | 22.3 | 0.44 | 0.63 | 0.19 |
| 48 | 7749.32 | 19.97 | −0.41 | 0.09 | 0.5 |
| 49 | 7760.02 | 28.72 | 0.46 | 0.71 | 0.25 |
| 50 | 10671.25 | 28.92 | 0.48 | 0.67 | 0.19 |

12. The method according to claim 10, wherein an evaluation of the amplitude of markers 51 to 279 is effected by using the following reference values:

| | | |
|---|---|---|
| | | Mean amplitude per |
| Mass | Migration | group |
| No. | [g/mol] | time [min] | Alzheimer | Controls |
|---|---|---|---|---|
| 51 | 824.48 | 19.92 | 568.85 | 501.97 |
| 52 | 840.49 | 19.92 | 810.27 | 593.64 |
| 53 | 846.38 | 25.96 | 100.62 | 122.25 |
| 54 | 897.41 | 25.86 | 81.54 | 48.26 |
| 55 | 911.3 | 34.1 | 242.62 | 247.96 |
| 56 | 950.57 | 24.13 | 78.07 | 67.87 |
| 57 | 973.26 | 35.47 | 179.96 | 150.04 |
| 58 | 984.45 | 26.32 | 110.69 | 132.44 |
| 59 | 988.57 | 35.65 | 211.75 | 188.31 |
| 60 | 1013.43 | 25.18 | 175.63 | 104.85 |
| 61 | 1016.3 | 35.53 | 909.17 | 810.15 |
| 62 | 1041.58 | 36.79 | 86.97 | 139.71 |
| 63 | 1053.52 | 25.25 | 87.43 | 46.52 |
| 64 | 1055.61 | 25.7 | 277.17 | 465.99 |
| 65 | 1082.55 | 23.75 | 78.89 | 50.53 |
| 66 | 1082.65 | 26.15 | 112.48 | 104.55 |
| 67 | 1083.5 | 27.14 | 118.51 | 108.08 |
| 68 | 1083.55 | 25.61 | 271.44 | 164.33 |
| 69 | 1085.63 | 36.39 | 140.8 | 145.98 |
| 70 | 1094.61 | 25.99 | 460.11 | 418.75 |
| 71 | 1130.37 | 35.19 | 670.41 | 372.37 |
| 72 | 1130.63 | 27.07 | 115.03 | 78.26 |
| 73 | 1135.56 | 27.03 | 60.46 | 58.13 |
| 74 | 1150.61 | 26.55 | 80.41 | 45.34 |
| 75 | 1158.62 | 27.02 | 68.79 | 40.98 |
| 76 | 1183.66 | 27.49 | 110.3 | 73.65 |
| 77 | 1186.56 | 27.21 | 315.86 | 221.73 |
| 78 | 1217.56 | 21.8 | 90.54 | 80.33 |
| 79 | 1229.62 | 27.38 | 99.75 | 62.81 |
| 80 | 1250.61 | 27.59 | 455.17 | 151.8 |
| 81 | 1276.43 | 35.7 | 1739.68 | 1000.12 |
| 82 | 1283.4 | 35.91 | 104.76 | 53 |
| 83 | 1300.67 | 29.7 | 120.17 | 58.65 |
| 84 | 1306.74 | 22.16 | 552.66 | 320.23 |
| 85 | 1333.44 | 35.93 | 268.17 | 204.63 |
| 86 | 1342.44 | 35.43 | 275.61 | 325.49 |
| 87 | 1347.71 | 28.94 | 83.29 | 67.88 |
| 88 | 1349.67 | 28.25 | 363.22 | 580.39 |
| 89 | 1360.67 | 28.06 | 90.7 | 94.56 |
| 90 | 1386.68 | 28.62 | 366.31 | 404.69 |
| 91 | 1388.74 | 27.97 | 319.52 | 394.09 |
| 92 | 1399.44 | 36.06 | 179.15 | 82.16 |
| 93 | 1404.73 | 29.49 | 165.77 | 108.43 |
| 94 | 1409.63 | 22.11 | 288.37 | 317.52 |
| 95 | 1449.7 | 22.04 | 77.35 | 102.04 |
| 96 | 1451.72 | 28.84 | 111.94 | 52.91 |
| 97 | 1464.7 | 30.57 | 1169.16 | 1453.9 |
| 98 | 1464.7 | 30.57 | 1346.04 | 1453.9 |
| 99 | 1475.77 | 30.05 | 216.71 | 146.11 |
| 100 | 1499.74 | 29.89 | 104.53 | 57.36 |
| 101 | 1519.76 | 31.87 | 392.54 | 271.33 |
| 102 | 1535.74 | 30 | 2366.44 | 3661.09 |
| 103 | 1542.78 | 23.24 | 713.91 | 533.83 |
| 104 | 1550.67 | 27.47 | 576.53 | 637.58 |
| 105 | 1552.61 | 30.78 | 176.95 | 110.5 |
| 106 | 1590.81 | 32.46 | 1491.15 | 1260.68 |
| 107 | 1596.83 | 30.32 | 873.36 | 577.23 |
| 108 | 1614.84 | 31.54 | 688.7 | 410.65 |
| 109 | 1623.65 | 31.1 | 697.2 | 364.52 |
| 110 | 1628.8 | 20.75 | 304.52 | 326.72 |
| 111 | 1636.79 | 22.71 | 1006.43 | 814.9 |
| 112 | 1659.81 | 27.41 | 589.62 | 358.41 |
| 113 | 1692.91 | 31.38 | 127.4 | 95.57 |
| 114 | 1703.89 | 33.01 | 287.84 | 258.89 |
| 115 | 1727.92 | 32.13 | 552.58 | 276.12 |
| 116 | 1732.88 | 31.36 | 596.19 | 543.08 |
| 117 | 1738.68 | 32.16 | 92.84 | 40.42 |
| 118 | 1741.83 | 30.56 | 114.01 | 84.13 |
| 119 | 1756.89 | 19.81 | 176.68 | 85.89 |
| 120 | 1771.97 | 30.02 | 328.73 | 214.56 |
| 121 | 1800.02 | 25 | 516.9 | 674.37 |
| 122 | 1816.98 | 33.53 | 395.82 | 180.33 |
| 123 | 1820.06 | 32.23 | 107.09 | 76.57 |
| 124 | 1841.77 | 36.26 | 847.46 | 1396.48 |
| 125 | 1849.68 | 18.31 | 405.84 | 688.91 |
| 126 | 1850.96 | 31.97 | 116.29 | 133.34 |
| 127 | 1867.72 | 33.07 | 1356.26 | 956.64 |
| 128 | 1869.92 | 31.28 | 223.93 | 140.79 |
| 129 | 1873 | 21.26 | 440.7 | 270.85 |
| 130 | 1900.07 | 24.19 | 920.26 | 373.01 |
| 131 | 1914.07 | 25.67 | 1519.39 | 1746.72 |
| 132 | 1915.72 | 34.24 | 180.37 | 151.24 |
| 133 | 1917.03 | 24.99 | 197.09 | 216.02 |
| 134 | 1955.95 | 27.81 | 129.24 | 99.09 |
| 135 | 2021.11 | 21.85 | 346.14 | 257.95 |
| 136 | 2041.99 | 32.56 | 193.08 | 127.84 |
| 137 | 2053.18 | 20.54 | 501.38 | 674.91 |
| 138 | 2072.15 | 27.04 | 601.86 | 494.89 |
| 139 | 2077.07 | 25.44 | 261.1 | 214.6 |
| 140 | 2085.12 | 33.75 | 215.62 | 302.81 |
| 141 | 2140.12 | 20.56 | 256.95 | 290.52 |
| 142 | 2166.09 | 27.84 | 128.57 | 52.54 |
| 143 | 2168.66 | 34.88 | 257.1 | 291.77 |
| 144 | 2179.03 | 25.31 | 84.93 | 31.65 |
| 145 | 2278.11 | 26.17 | 438.37 | 314.65 |
| 146 | 2283.18 | 26.31 | 619.59 | 634.52 |
| 147 | 2314.3 | 22.71 | 1018.08 | 1167.69 |
| 148 | 2320.43 | 23.21 | 212.67 | 166.8 |
| 149 | 2327.24 | 34.9 | 1737.42 | 1517.73 |
| 150 | 2341.12 | 26.74 | 304.17 | 535.15 |
| 151 | 2343.24 | 34.95 | 270.79 | 236.16 |
| 152 | 2368.24 | 21.24 | 101.48 | 105.72 |
| 153 | 2377.25 | 28.07 | 2711.11 | 1749.4 |
| 154 | 2390.26 | 27.33 | 617.62 | 500.67 |
| 155 | 2392.73 | 35.48 | 512.31 | 482.03 |
| 156 | 2409.28 | 27.64 | 275.81 | 159.58 |
| 157 | 2410.3 | 30.62 | 338.59 | 232.89 |
| 158 | 2448.39 | 21.07 | 488.24 | 440.74 |
| 159 | 2475.21 | 27.09 | 757.06 | 535.45 |
| 160 | 2475.27 | 24.74 | 478.15 | 297.04 |
| 161 | 2486.04 | 35.76 | 2280.47 | 1824.17 |
| 162 | 2500.33 | 19.99 | 110.01 | 94.92 |
| 163 | 2521.36 | 19.26 | 516.08 | 635.2 |
| 164 | 2522.35 | 27.9 | 4135.44 | 3306.09 |
| 165 | 2527.42 | 19.79 | 393.22 | 500.27 |
| 166 | 2549.29 | 27.66 | 99.25 | 108.48 |
| 167 | 2584.37 | 35.6 | 451.18 | 260.07 |
| 168 | 2684.32 | 21.38 | 1926.21 | 155.86 |
| 169 | 2698.41 | 22.52 | 182.49 | 220.4 |
| 170 | 2799.17 | 25.07 | 445.75 | 375.83 |
| 171 | 2816.31 | 28.74 | 1366.84 | 1928.38 |
| 172 | 2851.42 | 27.7 | 495.9 | 288.67 |
| 173 | 2923.51 | 36.73 | 818.47 | 883.22 |
| 174 | 2982.72 | 19.97 | 3750.78 | 5523.72 |
| 175 | 3019.54 | 24.72 | 170.57 | 189.58 |
| 176 | 3173.68 | 22.95 | 155.36 | 252.28 |
| 177 | 3192.67 | 37.66 | 689.53 | 1089.16 |
| 178 | 3215.61 | 28.53 | 1305.23 | 2016.28 |
| 179 | 3218.4 | 30.31 | 1384.58 | 1875.79 |
| 180 | 3221.59 | 22.7 | 170.45 | 217.24 |
| 181 | 3279.66 | 25.32 | 141.5 | 83 |
| 182 | 3282.34 | 36.05 | 1082.36 | 1351.09 |
| 183 | 3298.42 | 36.17 | 268.81 | 282.69 |
| 184 | 3302.68 | 23.31 | 316.81 | 508.67 |
| 185 | 3324.7 | 21.62 | 422.39 | 490.7 |
| 186 | 3386.77 | 22.21 | 670.34 | 395.98 |
| 187 | 3401.73 | 23.48 | 2618.95 | 4622.56 |
| 188 | 3408.76 | 26.04 | 175.22 | 94.81 |
| 189 | 3462.68 | 21.25 | 2161.32 | 2653.08 |
| 190 | 3466.75 | 23.9 | 904.51 | 559.21 |
| 191 | 3524.8 | 31.72 | 1130.3 | 410.29 |
| 192 | 3529.84 | 23.7 | 1979.26 | 2916.13 |
| 193 | 3595.77 | 23.89 | 344.53 | 455.24 |
| 194 | 3605.4 | 21.53 | 321.29 | 479.63 |
| 195 | 3614.82 | 22.01 | 161.46 | 267.85 |
| 196 | 3644.79 | 29.39 | 1038.75 | 749.16 |
| 197 | 3645.25 | 26.69 | 1161.31 | 741.33 |
| 198 | 3650.74 | 19.9 | 1029.39 | 1161.19 |
| 199 | 3672.71 | 21.9 | 1896.48 | 819.91 |
| 200 | 3677.74 | 24.3 | 1717.39 | 1567.09 |
| 201 | 3685.86 | 22.21 | 9259.98 | 16077.47 |
| 202 | 3687.78 | 20.58 | 832.51 | 1095.77 |
| 203 | 3693.93 | 27.15 | 344.49 | 218.63 |
| 204 | 3741.63 | 20.1 | 547.74 | 806.46 |
| 205 | 3768.77 | 31.84 | 533.93 | 377.59 |
| 206 | 3782.94 | 22.68 | 360.79 | 364.04 |
| 207 | 3788.04 | 25.24 | 609.58 | 782.85 |
| 208 | 3792.76 | 27.25 | 682.32 | 432.09 |
| 209 | 3819.82 | 32.74 | 120.7 | 133.76 |
| 210 | 3881.87 | 24.56 | 3257.71 | 1968.09 |
| 211 | 3903.08 | 25.71 | 2177.14 | 2780.86 |
| 212 | 3905.8 | 27.72 | 23257.92 | 16304.43 |
| 213 | 3929.73 | 23.07 | 2742.02 | 3430.19 |
| 214 | 3934.76 | 24.02 | 653.19 | 487.54 |
| 215 | 3951.07 | 22.77 | 25509.01 | 32844.44 |
| 216 | 4005.54 | 22.57 | 566.89 | 387.06 |
| 217 | 4087.12 | 26.92 | 607.44 | 490.16 |
| 218 | 4112.78 | 24.46 | 267.94 | 308.15 |
| 219 | 4131.68 | 22.54 | 653.71 | 586.61 |
| 220 | 4164.86 | 25.11 | 781.59 | 466.12 |
| 221 | 4192.99 | 34.95 | 1761.93 | 1496.61 |
| 222 | 4203.13 | 26.14 | 613.9 | 1051.15 |
| 223 | 4218.31 | 20.6 | 939.4 | 1360.44 |
| 224 | 4232.32 | 32.35 | 318.48 | 349.1 |
| 225 | 4250 | 27.68 | 504.35 | 286.25 |
| 226 | 4278 | 25.3 | 1161.81 | 745.21 |
| 227 | 4301.93 | 21.26 | 476.37 | 698.84 |
| 228 | 4307.09 | 31.58 | 157.37 | 88.2 |
| 229 | 4349.06 | 28.17 | 15449.01 | 7992.31 |
| 230 | 4384.07 | 19.8 | 465.18 | 981.39 |
| 231 | 4428.79 | 32.86 | 827.48 | 433.08 |
| 232 | 4464.64 | 21.54 | 4084.32 | 2311.8 |
| 233 | 4468.61 | 23.47 | 3429.99 | 2945.88 |
| 234 | 4510.97 | 28.56 | 1157.68 | 629.08 |
| 235 | 4548.03 | 25.95 | 3177.96 | 2703.21 |
| 236 | 4562 | 29.31 | 509.94 | 403.2 |
| 237 | 4562 | 29.31 | 574.68 | 403.2 |
| 238 | 4564.18 | 26.01 | 497.67 | 438.34 |
| 239 | 4583.84 | 24.2 | 7608.37 | 7241.51 |
| 240 | 4637.81 | 27.82 | 337.27 | 294.89 |
| 241 | 4730.71 | 19.63 | 797.93 | 1759.4 |
| 242 | 4749.41 | 21.81 | 725.74 | 426.89 |
| 243 | 4750.06 | 19.53 | 4365.88 | 3189.94 |
| 244 | 4890.59 | 23.27 | 1965.12 | 1418.61 |
| 245 | 4933.64 | 20.57 | 878.93 | 735.48 |
| 246 | 4986.98 | 21.35 | 637.73 | 533.05 |
| 247 | 5039.63 | 25.68 | 1039.64 | 1389.17 |
| 248 | 5042.1 | 24.31 | 3686.99 | 3120.74 |
| 249 | 5060.4 | 20.09 | 3307.86 | 1942.65 |
| 250 | 5110.36 | 25.28 | 637.44 | 697.57 |
| 251 | 5122.44 | 20.75 | 1342 | 851.23 |
| 252 | 5157.79 | 34.26 | 200.52 | 208.01 |
| 253 | 5181.63 | 25.36 | 5716.39 | 4305.72 |
| 254 | 5258.56 | 22.09 | 12476.68 | 10602.4 |
| 255 | 5439.46 | 25.41 | 713.92 | 522.3 |
| 256 | 5495.33 | 31.72 | 1776.93 | 1297.43 |
| 257 | 5660.6 | 31.55 | 893.02 | 595.49 |
| 258 | 5745.2 | 19.72 | 851.4 | 1608.76 |
| 259 | 5891.27 | 24.16 | 482.2 | 422.63 |
| 260 | 6224.17 | 25.27 | 1104.36 | 995.62 |
| 261 | 6327.46 | 22.03 | 1410.46 | 1027.79 |
| 262 | 6498.75 | 20.1 | 3989.56 | 2972.62 |
| 263 | 6611.97 | 28.44 | 311.21 | 338.55 |
| 264 | 6617.76 | 24.03 | 2799.82 | 1943.58 |
| 265 | 6813.98 | 23.14 | 4217.01 | 3699.9 |
| 266 | 6881.81 | 21.72 | 450.1 | 868.21 |
| 267 | 6893.8 | 24.57 | 2537.57 | 1569.65 |
| 268 | 6985.31 | 21.87 | 2238.63 | 1577.04 |
| 269 | 7026.99 | 22.02 | 2132.66 | 1480.92 |
| 270 | 7049.04 | 22.79 | 8522.63 | 6229.31 |
| 271 | 7106.83 | 23 | 766.91 | 575.59 |
| 272 | 7142.49 | 21.32 | 1014.86 | 557.95 |
| 273 | 7210.86 | 22.94 | 915.21 | 568.24 |
| 274 | 8176.16 | 19.47 | 11495.92 | 7835.63 |
| 275 | 8186.93 | 20.72 | 1706.07 | 1130.11 |
| 276 | 8289.28 | 19.39 | 4317.47 | 3227.21 |
| 277 | 9724.01 | 25.64 | 3380.24 | 1957.82 |
| 278 | 12529 | 20.64 | 38234.95 | 9397.12 |
| 279 | 14557.83 | 19.21 | 1514.79 | 2225.19 |

13. The method according to claim 10, wherein at least three or at least four or at least five or at least ten or all polypeptide markers as defined in claim 1 are used.

14. The method according to claim 10, wherein said sample from an individual is a blood (serum or plasma) sample or a cerebrospinal fluid sample.

15. The method according to claim 10, wherein capillary electrophoresis, HPLC, gas-phase ion spectrometry and/or mass spectrometry is used for detecting the presence or absence of the polypeptide marker or markers.

16. The method according to claim 10, wherein a capillary electrophoresis is performed before the molecular mass of the polypeptide markers is measured.

17. The method according to claim 10, wherein mass spectrometry is used for detecting the presence or absence of the polypeptide marker or markers.

18. Use of at least one polypeptide marker selected from marker Nos. 1 to 279 and characterized by the following values for the molecular masses and migration times:

| | Mass | Migration |
|---|---|---|
| No. | [g/mol] | time [min] |
| 1 | 1073.35 | 34.96 |
| 2 | 1073.35 | 35.9 |
| 3 | 1110.31 | 37.67 |
| 4 | 1111.3 | 34.97 |
| 5 | 1196.36 | 36.37 |
| 6 | 1265.63 | 27.22 |
| 7 | 1322.44 | 36.2 |
| 8 | 1387.07 | 20.42 |
| 9 | 1390.49 | 36.26 |
| 10 | 1423.57 | 29.85 |
| 11 | 1453.55 | 30.47 |
| 12 | 1490.43 | 35.48 |
| 13 | 1494.62 | 30.23 |
| 14 | 1819.86 | 23.51 |
| 15 | 1927.02 | 21 |
| 16 | 2128.97 | 26.03 |
| 17 | 2214.22 | 26.48 |
| 18 | 2313.26 | 30.32 |
| 19 | 2423.19 | 27.03 |
| 20 | 2428.11 | 27.16 |
| 21 | 2521.33 | 27.86 |
| 22 | 2644.31 | 21.1 |
| 23 | 2706.37 | 28.3 |
| 24 | 3068.46 | 30.03 |
| 25 | 3079.61 | 37.02 |
| 26 | 3153.38 | 35.65 |
| 27 | 3229.79 | 25.02 |
| 28 | 3438.77 | 23.49 |
| 29 | 3481.8 | 30.62 |
| 30 | 3567.61 | 23.88 |
| 31 | 3685.85 | 25.47 |
| 32 | 3921.84 | 29.49 |
| 33 | 4036.3 | 24.92 |
| 34 | 4038.22 | 20.53 |
| 35 | 4093.96 | 21.04 |
| 36 | 4145.22 | 24.51 |
| 37 | 4372.3 | 19.37 |
| 38 | 4377.95 | 30.81 |
| 39 | 4496.98 | 19.35 |
| 40 | 4516 | 19.37 |
| 41 | 4540.4 | 22 |
| 42 | 4586.98 | 19.38 |
| 43 | 4596.23 | 19.6 |
| 44 | 4810.95 | 19.77 |
| 45 | 5227.91 | 25.08 |
| 46 | 5582.16 | 24.3 |
| 47 | 6402.73 | 22.3 |
| 48 | 7749.32 | 19.97 |
| 49 | 7760.02 | 28.72 |
| 50 | 10671.25 | 28.92 |
| 51 | 824.48 | 19.92 |
| 52 | 840.49 | 19.92 |
| 53 | 846.38 | 25.96 |
| 54 | 897.41 | 25.86 |
| 55 | 911.3 | 34.1 |
| 56 | 950.57 | 24.13 |
| 57 | 973.26 | 35.47 |
| 58 | 984.45 | 26.32 |
| 59 | 988.57 | 35.65 |
| 60 | 1013.43 | 25.18 |
| 61 | 1016.3 | 35.53 |
| 62 | 1041.58 | 36.79 |
| 63 | 1053.52 | 25.25 |
| 64 | 1055.61 | 25.7 |
| 65 | 1082.55 | 23.75 |
| 66 | 1082.65 | 26.15 |
| 67 | 1083.5 | 27.14 |
| 68 | 1083.55 | 25.61 |
| 69 | 1085.63 | 36.39 |
| 70 | 1094.61 | 25.99 |
| 71 | 1130.37 | 35.19 |
| 72 | 1130.63 | 27.07 |
| 73 | 1135.56 | 27.03 |
| 74 | 1150.61 | 26.55 |
| 75 | 1158.62 | 27.02 |
| 76 | 1183.66 | 27.49 |
| 77 | 1186.56 | 27.21 |
| 78 | 1217.56 | 21.8 |
| 79 | 1229.62 | 27.38 |
| 80 | 1250.61 | 27.59 |
| 81 | 1276.43 | 35.7 |
| 82 | 1283.4 | 35.91 |
| 83 | 1300.67 | 29.7 |
| 84 | 1306.74 | 22.16 |
| 85 | 1333.44 | 35.93 |
| 86 | 1342.44 | 35.43 |
| 87 | 1347.71 | 28.94 |
| 88 | 1349.67 | 28.25 |
| 89 | 1360.67 | 28.06 |
| 90 | 1386.68 | 28.62 |
| 91 | 1388.74 | 27.97 |
| 92 | 1399.44 | 36.06 |
| 93 | 1404.73 | 29.49 |
| 94 | 1409.63 | 22.11 |
| 95 | 1449.7 | 22.04 |
| 96 | 1451.72 | 28.84 |
| 97 | 1464.7 | 30.57 |
| 99 | 1475.77 | 30.05 |
| 100 | 1499.74 | 29.89 |
| 101 | 1519.76 | 31.87 |
| 102 | 1535.74 | 30 |
| 103 | 1542.78 | 23.24 |
| 104 | 1550.67 | 27.47 |
| 105 | 1552.61 | 30.78 |
| 106 | 1590.81 | 32.46 |
| 107 | 1596.83 | 30.32 |
| 108 | 1614.84 | 31.54 |
| 109 | 1623.65 | 31.1 |
| 110 | 1628.8 | 20.75 |
| 111 | 1636.79 | 22.71 |
| 112 | 1659.81 | 27.41 |
| 113 | 1692.91 | 31.38 |
| 114 | 1703.89 | 33.01 |
| 115 | 1727.92 | 32.13 |
| 116 | 1732.88 | 31.36 |
| 117 | 1738.68 | 32.16 |
| 118 | 1741.83 | 30.56 |
| 119 | 1756.89 | 19.81 |
| 120 | 1771.97 | 30.02 |
| 121 | 1800.02 | 25 |
| 122 | 1816.98 | 33.53 |
| 123 | 1820.06 | 32.23 |
| 124 | 1841.77 | 36.26 |
| 125 | 1849.68 | 18.31 |
| 126 | 1850.96 | 31.97 |
| 127 | 1867.72 | 33.07 |
| 128 | 1869.92 | 31.28 |
| 129 | 1873 | 21.26 |
| 130 | 1900.07 | 24.19 |
| 131 | 1914.07 | 25.67 |
| 132 | 1915.72 | 34.24 |
| 133 | 1917.03 | 24.99 |
| 134 | 1955.95 | 27.81 |
| 135 | 2021.11 | 21.85 |
| 136 | 2041.99 | 32.56 |
| 137 | 2053.18 | 20.54 |
| 138 | 2072.15 | 27.04 |
| 139 | 2077.07 | 25.44 |
| 140 | 2085.12 | 33.75 |
| 141 | 2140.12 | 20.56 |
| 142 | 2166.09 | 27.84 |
| 143 | 2168.66 | 34.88 |
| 144 | 2179.03 | 25.31 |
| 145 | 2278.11 | 26.17 |
| 146 | 2283.18 | 26.31 |
| 147 | 2314.3 | 22.71 |
| 148 | 2320.43 | 23.21 |
| 149 | 2327.24 | 34.9 |
| 150 | 2341.12 | 26.74 |
| 151 | 2343.24 | 34.95 |
| 152 | 2368.24 | 21.24 |
| 153 | 2377.25 | 28.07 |
| 154 | 2390.26 | 27.33 |
| 155 | 2392.73 | 35.48 |
| 156 | 2409.28 | 27.64 |
| 157 | 2410.3 | 30.62 |
| 158 | 2448.39 | 21.07 |
| 159 | 2475.21 | 27.09 |
| 160 | 2475.27 | 24.74 |
| 161 | 2486.04 | 35.76 |
| 162 | 2500.33 | 19.99 |
| 163 | 2521.36 | 19.26 |
| 164 | 2522.35 | 27.9 |
| 165 | 2527.42 | 19.79 |
| 166 | 2549.29 | 27.66 |
| 167 | 2584.37 | 35.6 |
| 168 | 2684.32 | 21.38 |
| 169 | 2698.41 | 22.52 |
| 170 | 2799.17 | 25.07 |
| 171 | 2816.31 | 28.74 |
| 172 | 2851.42 | 27.7 |
| 173 | 2923.51 | 36.73 |
| 174 | 2982.72 | 19.97 |
| 175 | 3019.54 | 24.72 |
| 176 | 3173.68 | 22.95 |
| 177 | 3192.67 | 37.66 |
| 178 | 3215.61 | 28.53 |
| 179 | 3218.4 | 30.31 |
| 180 | 3221.59 | 22.7 |
| 181 | 3279.66 | 25.32 |
| 182 | 3282.34 | 36.05 |
| 183 | 3298.42 | 36.17 |
| 184 | 3302.68 | 23.31 |
| 185 | 3324.7 | 21.62 |
| 186 | 3386.77 | 22.21 |
| 187 | 3401.73 | 23.48 |
| 188 | 3408.76 | 26.04 |
| 189 | 3462.68 | 21.25 |
| 190 | 3466.75 | 23.9 |
| 191 | 3524.8 | 31.72 |
| 192 | 3529.84 | 23.7 |
| 193 | 3595.77 | 23.89 |
| 194 | 3605.4 | 21.53 |
| 195 | 3614.82 | 22.01 |
| 196 | 3644.79 | 29.39 |
| 197 | 3645.25 | 26.69 |
| 198 | 3650.74 | 19.9 |
| 199 | 3672.71 | 21.9 |
| 200 | 3677.74 | 24.3 |
| 201 | 3685.86 | 22.21 |
| 202 | 3687.78 | 20.58 |
| 203 | 3693.93 | 27.15 |
| 204 | 3741.63 | 20.1 |
| 205 | 3768.77 | 31.84 |
| 206 | 3782.94 | 22.68 |
| 207 | 3788.04 | 25.24 |
| 208 | 3792.76 | 27.25 |
| 209 | 3819.82 | 32.74 |
| 210 | 3881.87 | 24.56 |
| 211 | 3903.08 | 25.71 |
| 212 | 3905.8 | 27.72 |
| 213 | 3929.73 | 23.07 |
| 214 | 3934.76 | 24.02 |
| 215 | 3951.07 | 22.77 |
| 216 | 4005.54 | 22.57 |
| 217 | 4087.12 | 26.92 |
| 218 | 4112.78 | 24.46 |
| 219 | 4131.68 | 22.54 |
| 220 | 4164.86 | 25.11 |
| 221 | 4192.99 | 34.95 |
| 222 | 4203.13 | 26.14 |
| 223 | 4218.31 | 20.6 |
| 224 | 4232.32 | 32.35 |
| 225 | 4250 | 27.68 |
| 226 | 4278 | 25.3 |
| 227 | 4301.93 | 21.26 |
| 228 | 4307.09 | 31.58 |
| 229 | 4349.06 | 28.17 |
| 230 | 4384.07 | 19.8 |
| 231 | 4428.79 | 32.86 |
| 232 | 4464.64 | 21.54 |
| 233 | 4468.61 | 23.47 |
| 234 | 4510.97 | 28.56 |
| 235 | 4548.03 | 25.95 |
| 236 | 4562 | 29.31 |
| 237 | 4562 | 29.31 |
| 238 | 4564.18 | 26.01 |
| 239 | 4583.84 | 24.2 |
| 240 | 4637.81 | 27.82 |
| 241 | 4730.71 | 19.63 |
| 242 | 4749.41 | 21.81 |
| 243 | 4750.06 | 19.53 |
| 244 | 4890.59 | 23.27 |
| 245 | 4933.64 | 20.57 |
| 246 | 4986.98 | 21.35 |
| 247 | 5039.63 | 25.68 |
| 248 | 5042.1 | 24.31 |
| 249 | 5060.4 | 20.09 |
| 250 | 5110.36 | 25.28 |
| 251 | 5122.44 | 20.75 |
| 252 | 5157.79 | 34.26 |
| 253 | 5181.63 | 25.36 |
| 254 | 5258.56 | 22.09 |
| 255 | 5439.46 | 25.41 |
| 256 | 5495.33 | 31.72 |
| 257 | 5660.6 | 31.55 |
| 258 | 5745.2 | 19.72 |
| 259 | 5891.27 | 24.16 |
| 260 | 6224.17 | 25.27 |
| 261 | 6327.46 | 22.03 |
| 262 | 6498.75 | 20.1 |
| 263 | 6611.97 | 28.44 |
| 264 | 6617.76 | 24.03 |
| 265 | 6813.98 | 23.14 |
| 266 | 6881.81 | 21.72 |
| 267 | 6893.8 | 24.57 |
| 268 | 6985.31 | 21.87 |
| 269 | 7026.99 | 22.02 |
| 270 | 7049.04 | 22.79 |
| 271 | 7106.83 | 23 |
| 272 | 7142.49 | 21.32 |
| 273 | 7210.86 | 22.94 |
| 274 | 8176.16 | 19.47 |
| 275 | 8186.93 | 20.72 |
| 276 | 8289.28 | 19.39 |
| 277 | 9724.01 | 25.64 |
| 278 | 12529 | 20.64 |
| 279 | 14557.83 | 19.21 |

for the diagnosis of Alzheimer's disease.

19. A combination of markers comprising at least 10 markers selected from markers 1 to 279, which are characterized by the molecular masses and migration times (CE times) according to claim 1.
